(19) United States
(12) Patent Application Publication
(10) Pub. No.: US 2019/0060249 A1
(43) Pub. Date: Feb. 28, 2019

(54) MONOTERPENE ACTIVATORS OF ALDEHYDE DEHYDROGENASE 3A1 AND METHODS OF USE THEREOF

(71) Applicant: The Board of Trustees of the Leland Stanford Junior University, Stanford, CA (US)

(72) Inventors: Julie Saiki, Stanford, CA (US); Che-Hong Chen, Fremont, CA (US); Quynh-Thu Le, Stanford, CA (US); Daria Mochly-Rosen, Menlo Park, CA (US)

(21) Appl. No.: 16/110,934

(22) Filed: Aug. 23, 2018

Related U.S. Application Data
(60) Provisional application No. 62/549,849, filed on Aug. 24, 2017.

(51) Int. Cl. A61K 31/015; A61P 27/02; A61P 35/00; C12N 5/071; C12N 5/0775

(52) U.S. Cl. CPC A61K 31/015; A61P 27/02; A61N 5/00; C12N 5/0633; C12N 5/0662; A61P 35/00

(57) ABSTRACT

The present disclosure provides methods of increasing proliferation of adult salivary stem cells, methods of protecting adult salivary stem cells and improving salivary gland function. The methods include contacting adult salivary stem cells in vivo, in vitro, or ex vivo with a therapeutically effective amount of at least one isolated monoterpene and subjecting the adult salivary stem cells to radiation treatment. Increasing proliferation of adult salivary stem cells can be carried out to provide for an increase in the number of adult salivary stem cells and improve salivary gland function in an individual undergoing radiotherapy for head and neck cancer. The methods also include treating an individual with dry eye with a therapeutically effective amount of at least one isolated monoterpene.

### CROSS-REFERENCE

This application claims the benefit of U.S. Provisional Patent Application No. 62/549,849, filed Aug. 24, 2017, which application is incorporated herein by reference in its entirety.

### STATEMENT REGARDING FEDERALLY SPONSORED RESEARCH

This invention was made with Government support under contracts AA011147, CA067166, CA180816, and DE025227 awarded by the National Institutes of Health. The Government has certain rights in the invention.

### INTRODUCTION

Drying of the mucous membranes such as in the mouth and eyes is a serious health problem. Most head and neck cancer (HNC) patients receive radiotherapy (RT) as part of their cancer management. Radiation exposure results in permanent damage to the salivary glands, causing loss of function and subsequent RT-related xerostomia or dry mouth. Patients with RT xerostomia experience reduced quality and quantity of saliva, which leads to considerable morbidities, including solid food dysphagia, chronic dental caries, recurrent oral infections and rare mandibular osteoradionecrosis. It is estimated that >60% of patients receiving head and neck RT suffer from many of these side effects. Current approved medical managements for RT xerostomia include the use of salivary substitutes, lubricants and cholinergic agonists to stimulate salivary secretion. These treatments remain palliative in nature, require chronic use and are often ineffective. Intensity modulated radiotherapy (IMRT) can protect the parotid glands from direct radiation injury in selective cases; however, it often cannot spare the submandibular glands (SMG), which are responsible for resting saliva production. The vicinity of the SMG to the level II nodes, which are the most commonly involved nodes in HNC, makes it harder to spare them from direct RT beams At least one randomized study indicated that although IMRT resulted in improved parotid sparing and more stimulatory saliva production, it did not result in significant improvement of patient's subjective xerostomia. In contrast, SMG transfer and sparing from direct RT beams was associated with a significantly better subjective xerostomia function as assessed by xerostomia and quality of life questionnaires.

Patients receiving RT for HNC, whose lacrimal apparatus is exposed to fractionated external beam RT, may also suffer from damage to the lacrimal gland, resulting in severe dry eye syndrome. Dry eye disease refers to a variety of conditions associated with abnormalities in the tear film and insufficient lubrication and/or moisture in the eye. Symptoms of dry eye disease include dryness, scratching, itching, burning, irritation, and a sandy-gritty feeling in the eye. Dry eye disease may also result in visual disturbance and tear film instability, with the potential for damage to the ocular surface. Dry eye disease may be associated with an increase in tear osmolality.

Despite widespread IMRT use in HNC, development of methods to reconstitute salivary gland tissue, specifically SMG, recovery of physiological salivary secretion after RT, and recovery of the lacrimal gland in the eye, is needed in HNC patients.

### SUMMARY

The present disclosure provides methods of increasing proliferation of adult salivary stem cells, methods of protecting adult salivary stem cells and improving salivary gland function. The methods include contacting adult salivary stem cells in vivo, in vitro, or ex vivo with a therapeutically effective amount of at least one isolated monoterpene and subjecting the adult salivary stem cells to radiation treatment. The radiation treatment can be carried out before or after contacting the adult salivary stem cells with the isolated monoterpene. Increasing proliferation of adult salivary stem cells can be carried out to provide for an increase in the number of adult salivary stem cells and improve salivary gland function in an individual undergoing radiotherapy for head and neck cancer.

Also provided herein are methods of treating dry eye. The methods include treating an individual with dry eye with a therapeutically effective amount of at least one isolated monoterpene.

### BRIEF DESCRIPTION OF THE DRAWINGS

FIG. 1A-1H are a collection of images and graphs illustrating that loss of ALDH3A1 impairs normal salivary stem/progenitor cell (SSPC) function and accelerates loss of saliva production after radiation.

FIG. 2A-2D are a collection of images and graphs illustrating the results of a natural product screen which identifies Alda-341 (D-limonene) as a novel small molecule activator of ALDH3A1.

FIG. 3A-3I are a collection of images and graphs illustrating that Alda-341 increases normal SSPC function by enhancing sphere formation and differentiation.

FIG. 4A-4J are a collection of images and graphs illustrating that Alda-341 reduces aldehydic load after radiation in isolated embryonic salivary glands and mitigates radiation-induced hyposalivation in vivo.

FIG. 5A-5C are a collection of graphs showing that Alda-341 is distributed to human salivary glands.

### DEFINITIONS

As used herein, the term “aldehyde dehydrogenase” or “ALDH” refers to an enzyme that oxidizes an aldehyde to its corresponding acid in an NAD+-dependent or an NADP+-dependent reaction. The term “ALDH” encompasses any of the known ALDH isozymes, including ALDH1, ALDH2, ALDH3, ALDH4, ALDH5, ALDH6, etc.

The term “aldehyde dehydrogenase 3 family, member A1” or “ALDH3A1” or “ALDH3” encompasses ALDH3 from various species. Amino acid sequences of ALDH3 from various species are publicly available. See, e.g., GenBank Accession Nos. AAB26658 (Homo sapiens ALDH3), NP_000683 (Homo sapiens ALDH3), P30838 (Homo sapiens ALDH3), NP_001106196 (Mus musculus ALDH3), and AAH70924 (Rattus norvegicus ALDH3). The term “ALDH3” as used herein also encompasses fragments, fusion proteins, and variants (e.g., variants having one or more amino acid substitutions, addition, deletions, and/or insertions) that retain ALDH3 enzymatic activity. The term “ALDH3” encompasses an aldehyde dehydrogenase that exhibits specificity toward aromatic aldehydes, e.g., oxidizing aromatic aldehydes of the 2-naphthaldehyde series, but inactive toward 1-naphthaldehydes and higher polyaromatic aldehydes. The term “ALDH3” encompasses an aldehyde dehydrogenase that can use both NAD+ and NADP+ as co-substrate. The term “ALDH3” encompasses aldehyde dehydrogenase found naturally in the stomach, in the lung, in saliva, and in the cornea.

The terms “protection” or “protecting adult salivary stem cells” as used herein refers to protection from radiation injury, e.g., preservation of salivary stem cells in radiation-induced xerostomia. The term may also refer to protection of salivary stem cells from damaging agents, e.g., damaging toxins, chemotherapy agents, radiation treatment and the like.

The term “improving salivary gland function” may refer to the improvement of all stem cell functions, such as salivary stem cell self-renewal and differentiation. It may also refer to reduced radiation injury.

As used herein, the term “increasing proliferation of adult salivary stem cells” or “expansion of adult salivary stem cells” refers to an increase in, or expansion of, the number of salivary stem cells relative to a starting population of salivary stem cells.

The terms “subject,” “individual,” and “patient” are used interchangeably herein to a member or members of any mammalian or non-mammalian species that may have a need for the pharmaceutical methods, compositions and treatments described herein. Subjects and patients thus include, without limitation, primate (including humans and non-human primates), canine, feline, ungulate (e.g., equine, bovine, swine (e.g., pig)), avian, and other subjects. Humans and non-human mammals having commercial importance (e.g., livestock and domesticated animals) are of particular interest.

“Mammal” refers to a member or members of any mammalian species, and includes, by way of example, canines; felines; equines; bovines; ovines; rodentia, etc. and primates, e.g., humans. Non-human animal models, particularly mammals, e.g. a non-human primate, a murine (e.g., a mouse, a rat), lagomorpha, etc. may be used for experimental investigations.

The term “isolated compound” means a compound which has been substantially separated from, or enriched relative to, other compounds with which it occurs in nature. Isolated compounds are at least about 80%, at least about 90% pure, at least about 98% pure, or at least about 99% pure, by weight. The present disclosure is meant to comprehend diastereomers as well as their racemic and resolved, enantiomerically pure forms and pharmaceutically acceptable salts thereof.

A “therapeutically effective amount” or “efficacious amount” means the amount of a compound that, when administered to a mammal or other subject for treating a disease or condition, is sufficient, in combination with another agent, or alone in one or more doses, to effect such treatment for the disease or condition. The “therapeutically effective amount” will vary depending on the compound, the disease and its severity and the age, weight, etc., of the subject to be treated.

A “pro-drug” means any compound that releases an active parent drug according to one or more of the generic formulas shown below in vivo when such pro-drug is administered to a mammalian subject or mammalian cells. Pro-drugs of a compound of one or more of the generic formulas shown below are prepared by modifying functional groups present in the compound of the generic formula in such a way that the modifications may be cleaved in vivo to release the parent compound. Pro-drugs include compounds of one or more of the generic formulas shown below wherein a hydroxy, amino, or sulfhydryl group in one or more of the generic formulas shown below is bonded to any group that may be cleaved in vivo to regenerate the free hydroxyl, amino, or sulfhydryl group, respectively. Examples of pro-drugs include, but are not limited to esters (e.g., acetate, formate, and benzoate derivatives), carbamates (e.g., N,N-dimethylaminocarbonyl) of hydroxy functional groups in compounds of one or more of the generic formulas shown below, and the like.

“Treating” or “treatment” of a condition or disease includes: (1) preventing at least one symptom of the conditions, i.e., causing a clinical symptom to not significantly develop in a mammal that may be exposed to or predisposed to the disease but does not yet experience or display symptoms of the disease, (2) inhibiting the disease, i.e., arresting or reducing the development of the disease or its symptoms, or (3) relieving the disease, i.e., causing regression of the disease or its clinical symptoms.

“In combination with,” or “co-administration,” as used herein, in the context of administering a first compound and at least a second compound, refers to uses where, for example, the first compound is administered during the entire course of administration of the second compound; where the first compound is administered for a period of time that is overlapping with the administration of the second compound, e.g. where administration of the first compound begins before the administration of the second compound and the administration of the first compound ends before the administration of the second compound ends; where the administration of the second compound begins before the administration of the first compound and the administration of the second compound ends before the administration of the first compound ends; where the administration of the first compound begins before administration of the second compound begins and the administration of the second compound ends before the administration of the first compound ends; where the administration of the second compound begins before administration of the first compound begins and the administration of the first compound ends before the administration of the second compound ends. As such, “in combination” can also refer to regimen involving administration of two or more compounds. “In combination with” as used herein also refers to administration of two or more compounds which may be administered in the same or different formulations, by the same of different routes, and in the same or different dosage form type.

The term “unit dosage form,” as used herein, refers to physically discrete units suitable as unitary dosages for human and animal subjects, each unit containing a predetermined quantity of a compound, calculated in an amount sufficient to produce the desired effect in association with a pharmaceutically acceptable diluent, carrier or vehicle. The specifications for a dosage form may depend on the particular compound employed and the effect to be achieved, and the pharmacodynamics associated with each compound in the host.

The term “physiological conditions” is meant to encompass those conditions compatible with living cells, e.g., predominantly aqueous conditions of a temperature, pH, salinity, etc. that are compatible with living cells.

A “pharmaceutically acceptable excipient,” “pharmaceutically acceptable diluent,” “pharmaceutically acceptable carrier,” and “pharmaceutically acceptable adjuvant” means an excipient, diluent, carrier, and adjuvant that are useful in preparing a pharmaceutical composition that are generally safe, non-toxic and neither biologically nor otherwise undesirable, and include an excipient, diluent, carrier, and adjuvant that are acceptable for veterinary use as well as human pharmaceutical use. “A pharmaceutically acceptable excipient, diluent, carrier and adjuvant” as used in the specification and claims includes one and more than one such excipient, diluent, carrier, and adjuvant.

As used herein, a “pharmaceutical composition” is meant to encompass a composition suitable for administration to a subject, such as a mammal, especially a human. In general, a “pharmaceutical composition” is sterile, and is free of contaminants that are capable of eliciting an undesirable response within the subject (e.g., the compound(s) in the pharmaceutical composition is pharmaceutical grade). Pharmaceutical compositions can be designed for administration to subjects or patients in need thereof via a number of different routes of administration including oral, buccal, rectal, parenteral, intraperitoneal, intradermal, intratracheal and the like. In some embodiments the composition is suitable for administration by a transdermal route, using a penetration enhancer other than dimethylsulfoxide (DMSO). In other embodiments, the pharmaceutical compositions are suitable for administration by a route other than transdermal administration. A pharmaceutical composition will in some embodiments include a compound (e.g., an ALDH agonist) and a pharmaceutically acceptable excipient. In some embodiments, a pharmaceutically acceptable excipient is other than DMSO.

As used herein, “pharmaceutically acceptable derivatives” of a compound of the invention include salts, esters, enol ethers, enol esters, acetals, ketals, orthoesters, hemiacetals, hemiketals, acids, bases, solvates, hydrates or prodrugs thereof. Such derivatives may be readily prepared by those of skill in this art using known methods for such derivatization. The compounds produced may be administered to animals or humans without substantial toxic effects and are either pharmaceutically active or are prodrugs.

A “pharmaceutically acceptable salt” of a compound means a salt that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

A “pharmaceutically acceptable ester” of a compound of the invention means an ester that is pharmaceutically acceptable and that possesses the desired pharmacological activity of the parent compound, and includes, but is not limited to, alkyl, alkenyl, alkynyl, aryl, heteroaryl, aralkyl, heteroaralkyl, cycloalkyl and heterocyclyl esters of acidic groups, including, but not limited to, carboxylic acids, phosphoric acids, phosphinic acids, sulfonic acids, sulfinic acids and boronic acids.

A “pharmaceutically acceptable enol ether” of a compound of the invention means an enol ether that is pharmaceutically acceptable and that possesses the desired pharmacological activity of the parent compound, and includes, but is not limited to, derivatives of formula C═C(OR) where R is hydrogen, alkyl, alkenyl, alkynyl, aryl, heteroaryl, aralkyl, heteroaralkyl, cycloalkyl or heterocyclyl.

A “pharmaceutically acceptable solvate or hydrate” of a compound of the invention means a solvate or hydrate complex that is pharmaceutically acceptable and that possesses the desired pharmacological activity of the parent compound, and includes, but is not limited to, complexes of a compound of the invention with one or more solvent or water molecules, or 1 to about 100, or 1 to about 10, or one to about 2, 3 or 4, solvent or water molecules.

Compounds that have the same molecular formula but differ in the nature or sequence of bonding of their atoms or the arrangement of their atoms in space are termed “isomers.” Isomers that differ in the arrangement of their atoms in space are termed “stereoisomers.” Stereoisomers that are not mirror images of one another are termed “diastereomers” and those that are non-superimposable mirror images of each other are termed “enantiomers.” When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture.”

A compound may possess one or more asymmetric centers; such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see, e.g., the discussion in Chapter 4 of “Advanced Organic Chemistry”, 4th edition J. March, John Wiley and Sons, New York, 1992).

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “an ALDH3 agonist” includes a plurality of such agonists and reference to “the adult salivary stem cell” includes reference to one or more such stem cells and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides methods of increasing proliferation of adult salivary stem cells, methods of protecting adult salivary stem cells and improving salivary gland function. The present disclosure provides methods of increasing the number of adult salivary stem cells in an individual undergoing radiotherapy. The individual is generally undergoing radiotherapy for head and neck cancer. The methods include contacting adult salivary stem cells in vivo, in vitro, or ex vivo with a therapeutically effective amount of at least one isolated monoterpene and subjecting the adult salivary stem cells to radiation treatment. The radiation treatment can be carried out before or after contacting the adult salivary stem cells with the isolated monoterpene. Contacting the adult salivary stem cells with a monoterpene before or after radiation treatment increases the number of adult salivary stem cells by 25% or more. The present disclosure also provides methods of treating dry eye. The methods include administering a therapeutically effective amount of at least one isolated monoterpene to an individual in need thereof.

### Methods of Treating Adult Salivary Cells

The present disclosure provides methods of treating adult salivary stem cells, the methods include contacting a starting population of adult salivary stem cells in vitro, in vivo, or ex vivo with a therapeutically effective amount of at least one isolated monoterpene; and subjecting the adult salivary stem cells to radiation treatment before or after said contacting, wherein the contacting results in one or more of increasing proliferation of the adult salivary stem cells, protecting the adult salivary stem cells from the radiation treatment and improving the salivary gland function of the adult salivary stem cells relative to the starting population of adult salivary stem cells.

The present disclosure provides methods of increasing proliferation of adult salivary stem cells, the methods include contacting a starting population of adult salivary stem cells in vivo, in vitro, or ex vivo with a therapeutically effective amount of at least one isolated monoterpene; and subjecting the adult salivary stem cells to radiation treatment before or after said contacting, wherein after the contacting and the radiation treatment the number of adult salivary stem cells increases by 25% or more.

According to one embodiment, a subject method involves contacting adult salivary stem cells in vitro, in vivo, or ex vivo with a therapeutically effective amount of at least one isolated monoterpene, and subjecting the adult salivary stem cells to radiation treatment before or after said contacting, wherein the contacting and radiation treatment increases the number of adult salivary stem cells by 30% or more, 40% or more, 50% or more, 75% or more, at least 2-fold, at least 2.5-fold, at least 5-fold, at least 10-fold, or more than 10-fold, compared to the number of adult salivary stem cells not contacted with an isolated monoterpene having undergone radiation treatment.

The present disclosure also provides methods of protecting adult salivary stem cells, the methods include contacting a starting population of adult salivary stem cells in vivo, in vitro, or ex vivo with a therapeutically effective amount of at least one isolated monoterpene; and subjecting the adult salivary stem cells to radiation treatment before or after said contacting, wherein after the contacting and the radiation treatment the adult salivary stem cells are protected from radiation injury relative to the starting population of adult salivary stem cells.

According to one embodiment, a subject method involves contacting adult salivary stem cells in vitro, in vivo, or ex vivo with a therapeutically effective amount of at least one isolated monoterpene, and subjecting the adult salivary stem cells to radiation treatment before or after said contacting, wherein the contacting and radiation treatment protects the adult salivary stem cells from radiation treatment by 30% or more, 40% or more, 50% or more, 75% or more, at least 2-fold, at least 2.5-fold, at least 5-fold, at least 10-fold, or more than 10-fold, compared to the starting population of adult salivary stem cells not contacted with an isolated monoterpene having undergone radiation treatment.

The present disclosure further provides methods of improving the salivary gland function of adult salivary stem cells, the methods include contacting a starting population of adult salivary stem cells in vivo, in vitro, or ex vivo with a therapeutically effective amount of at least one isolated monoterpene; and subjecting the adult salivary stem cells to radiation treatment before or after said contacting, wherein after the contacting and the radiation treatment the adult salivary stem cells have improved salivary gland function relative to the starting population of adult salivary stem cells.

According to one embodiment, a subject method involves contacting adult salivary stem cells in vitro, in vivo, or ex vivo with a therapeutically effective amount of at least one isolated monoterpene, and subjecting the adult salivary stem cells to radiation treatment before or after said contacting, wherein the contacting and radiation treatment improves the salivary gland function of the adult salivary stem cells by 30% or more, 40% or more, 50% or more, 75% or more, at least 2-fold, at least 2.5-fold, at least 5-fold, at least 10-fold, or more than 10-fold, compared to the salivary gland function of adult salivary stem cells not contacted with an isolated monoterpene having undergone radiation treatment.

Increasing the number of adult salivary stem cells, protecting the salivary stem cells and improving the salivary gland function of adult salivary stem cells are useful for treating an individual who has undergone, or is about to undergo, radiation treatment for a head and neck cancer. Treating the adult salivary stem cells in an individual provides for an increase in the number of adult salivary stem cells in the individual following radiation treatment, protection of the adult salivary stem cells in an individual following radiation treatment and also provides for an increase in the number of functional saliva-producing cells in the individual following radiation treatment. For example, a subject method provides for an increase in the number of functional saliva-producing cells in an individual following radiation treatment for a head and neck cancer of 25% or more, 30% or more, 35% or more, 40% or more, 45% or more, 50% or more, 75% or more, at least 2-fold, at least 2.5-fold, at least 5-fold, at least 10-fold, or more than 10-fold, compared to the number of functional saliva-producing cells in the individual in the absence of treatment with a subject method.

As noted above, in some cases, a subject method is carried out in vitro. Thus, e.g., adult salivary stem cells can be contacted with at least one isolated monoterpene in vitro to increase the number of adult salivary stem cells. In some cases, adult salivary stem cells can be contacted with at least one isolated monoterpene in vitro to protect the adult salivary stem cells. In other cases, the adult salivary stem cells can be contacted with at least one isolated monoterpene in vitro to improve the salivary gland function of the adult salivary stem cells.

In some cases, a subject method is carried out ex vivo, e.g., adult salivary stem cells are obtained from a donor individual, the adult salivary stem cells are treated ex vivo by contacting the adult salivary stem cells with at least one isolated monoterpene, to produce an ex vivo treated population of donor adult salivary stem cells. In some cases, the treatment of adult salivary stem cells with at least one isolated monoterpene ex vivo results in an expanded population of adult salivary stem cells. In some cases, the treatment of adult salivary stem cells with at least one isolated monoterpene ex vivo results in the protection of the adult salivary stem cells. In other cases, the treatment of adult salivary stem cells with at least one isolated monoterpene ex vivo results in improvement of the salivary gland function of the adult salivary stem cells. In some cases, the treatment of adult salivary stem cells with at least one isolated monoterpene ex vivo results in expansion, protection and improvement of the salivary gland function of the adult salivary stem cells. The ex vivo treated population of donor adult salivary stem cells is introduced into a recipient individual, e.g., an individual who has head and neck cancer who has undergone radiation treatment for the cancer. In some instances, the donor individual is the same as the recipient individual, e.g., adult salivary stem cells are obtained from the donor individual before the donor individual undergoes radiation treatment for a head and neck cancer, the adult salivary stem cells are treated ex vivo, as described herein, and the ex vivo treated donor salivary stem cell population is introduced into the donor individual (who is now the recipient) after the donor has undergone radiation treatment for the head and neck cancer. In other embodiments, the donor individual and the recipient individual are not the same individual. In certain cases, the recipient individual is a human. In some cases, both the donor individual and the recipient individual are human.

A subject method can be carried out in vivo. For example, a therapeutically effective amount of at least one isolated monoterpene is administered to an individual before and/or after radiation treatment for a head and neck cancer.

In one embodiment, the isolated monoterpene is an ALDH3 agonist.

### Isolation and Maintenance of Adult Salivary Stem Cells

A number of approaches for isolating and culturing adult salivary stem cells are known in the art, and any such method can be used to obtain adult salivary stem cells for use in a subject method. For example, human salivary gland tissue may be isolated and cultured as described by Szlavik et al. (Tissue Eng. Part A (2008) 14:1915-26); and Rotter N. et al. (Stem Cells Dev. (2008) 17:509-518), the disclosures of which are incorporated herein by reference in their entireties. Human salivary stem cells can be isolated from tissues such as the submandibular gland and/or the parotid gland.

According to one aspect of the present disclosure, salivary gland tissue isolated from a donor individual is minced and dissociated in an appropriate cell dissociation medium, centrifuged, filtered, and resuspended in a medium with one or more growth factors (e.g., epidermal growth factor (EGF), fibroblast growth factor 2 (FGF2), insulin-like growth factor-1 (IGF-1), and the like), antibiotics, and so forth to support maintenance and viability of the dissociated cells. Optionally, the stem cells are isolated or enriched from the primary cell suspension. This may be achieved by contacting the donor adult salivary stem cells in vitro with a reagent (e.g., an antibody) that specifically recognizes a marker associated with adult salivary stem cells, where contacting the donor adult salivary stem cells with the reagent is performed prior to contacting the donor adult salivary stem cells with the isolated monoterpene.

Useful markers for salivary stem cells include CD34, CD90, c-Kit, CD44, Nestin, CD49f, ALDH1, and combinations thereof. For example, human and mouse adult salivary stem cells may be isolated by selecting for CD34-positive and cKit-positive cells, respectively, e.g., using the EASYSEP™ positive selection kit (STEMCELL Technologies, Inc., Vancouver, BC). Detection of markers such as CD49f can be achieved using antibody specific for the marker, where the antibody can comprise a detectable label. Standard methods such as fluorescence activated cell sorting (FACS) can be used to isolate the cells. ALDH expression can be detected using ALDEFLUOR® aldehyde dehydrogenase fluorescent detection label. For example, ALDH converts the ALDH substrate, BAAA (BODIPY-aminoacetaldehyde), into the fluorescent product BAA (BODIPY-aminoacetate). Cells expressing high levels of ALDH become brightly fluorescent and can be identified using standard flow cytometry methods and/or isolated by cell sorting. See, e.g., Deng et al. (2010) PLoS One 5:e10277.

Isolation and culture of mouse salivary stem cells is described in, e.g., Lombaert et al. (2008) PLoS One 3:e2063. Isolation and culture of human salivary stem cells is also described in Lombaert et al. (2008) supra.

In certain aspects, adult salivary stem cells (isolated or otherwise) may be maintained in a culture medium prior to being contacted with the isolated monoterpene. For example, the cells may be maintained in a medium that includes one or more factors that prevents the salivary stem cells from differentiating into more specialized cells.

According to one embodiment, the donor adult salivary stem cells are obtained from an individual (e.g., having a head and neck cancer) prior to that individual undergoing a radiation treatment, e.g., radiotherapy to treat a head and neck cancer. In other aspects, the donor adult stem cells are obtained from an individual other than a recipient individual, e.g., an individual who neither has cancer nor is undergoing radiation treatment. In certain cases, recipient individual is a human In some cases, the donor adult stem cells are obtained from a human individual.

Contacting Adult Salivary Stem Cells with an Isolated Monoterpene In Vitro

As noted above, in some cases, a subject method is carried out in vitro. Methods of the present disclosure include contacting adult salivary stem cells in vitro with an isolated monoterpene, which monoterpene may be, e.g., an activator of ALDH3. In the case of contacting adult salivary stem cells with an isolated monoterpene in vitro, the cell culture medium may be supplemented with an effective amount of the monoterpene. The cell culture medium may be chosen such that the medium is compatible with the monoterpene, e.g., the monoterpene is stable and active in the medium. The medium may be supplemented with one or more components that enhance the stability and/or activity of the isolated monoterpene.

Contacting Adult Salivary Stem Cells with an Isolated Monoterpene Ex Vivo

In some cases, a subject method is carried out ex vivo, e.g., adult salivary stem cells are obtained from a donor individual, the adult salivary stem cells are treated ex vivo by contacting the adult salivary stem cells with at least one isolated monoterpene, to produce an ex vivo treated population of donor adult salivary stem cells. In some cases, the treatment of adult salivary stem cells with at least one isolated monoterpene ex vivo results in an expanded population of adult salivary stem cells. In some cases, the treatment of adult salivary stem cells with at least one isolated monoterpene ex vivo results in the protection of the adult salivary stem cells. In other cases, the treatment of adult salivary stem cells with at least one isolated monoterpene ex vivo results in improvement of the salivary gland function of the adult salivary stem cells. In some cases, the treatment of adult salivary stem cells with at least one isolated monoterpene ex vivo results in the expansion, protection and improvement of the salivary gland function of the adult salivary stem cells.

The ex vivo treated population of donor adult salivary stem cells is introduced into a recipient individual, e.g., an individual who has head and neck cancer who has undergone radiation treatment for the cancer. An ex vivo treated adult salivary stem cell population can be obtained by culturing adult salivary stem cells ex vivo in a culture medium comprising at least one isolated monoterpene, where the culturing can take place for about 4 hours to about 72 hours, e.g., from about 4 hours to about 8 hours, from about 8 hours to about 16 hours, from about 16 hours to about 24 hours, from about 24 hours to about 36 hours, from about 36 hours to about 48 hours, or from about 48 hours to about 72 hours, or more than 72 hours.

In some instances, the donor individual is the same as the recipient individual, in which case the cells are considered autologous. For example, adult salivary stem cells are obtained from the donor individual before the donor individual undergoes radiation treatment for a head and neck cancer, the adult salivary stem cells are expanded ex vivo, as described above, and the ex vivo expanded donor salivary stem cell population is introduced into the donor individual (who is now the recipient) after the donor has undergone radiation treatment for the head and neck cancer.

In other embodiments, the donor individual and the recipient individual are not the same individual, in which case the cells are allogeneic. The donor and the recipient can be human leukocyte antigen (HLA) typed before transplantation, and the closest HLA match identified as a suitable donor.

Introducing Salivary Stem Cells into a Recipient Individual

As noted above, methods of the present disclosure optionally include introducing a treated population of adult salivary stem cells (e.g., where the expansion, protection and improved salivary gland function is generated by contacting the cells with an isolated monoterpene, e.g., an ALDH3 agonist) into a recipient individual (e.g., a human), where the recipient individual has a head and neck cancer, and where the treated population of adult salivary stem cells are introduced after the recipient individual has undergone radiation treatment for the cancer. Introduction of the treated salivary stem cells is useful in a variety of applications, including stem cell-based therapies for restoring function to a salivary gland of an individual who suffers from xerostomia or dry mouth (e.g., radiotherapy-related xerostomia). For example, the introduced (e.g., “transplanted”) salivary stem cells may engraft to an irradiated salivary gland and differentiate into functional saliva-producing cells, thereby restoring or enhancing function of the irradiated salivary gland.

In one embodiment, the cells to be introduced into the recipient individual are provided as a suspension, which may be a single cell suspension, or a suspension of small clumps of cells, and which are distinguished from solid tissue grafts, which are implanted and are not injected or infused. The cell suspension is a form that can be injected or infused into a recipient. In another embodiment, the cells are provided as an ex vivo engineered tissue construct. Survival of the cells or tissue may be measured after short periods of time, e.g. after at least about three to about seven days.

The number of salivary stem cells transplanted into a recipient individual can vary from about 10 to about 10⁸, e.g., from 10 to 10², from about 10² to about 10³, from about 10³ to about 10⁴, from about 10⁴ to about 10⁵, from about 10⁵ to about 10⁶, from about 10⁶ to about 10⁷, or from about 10⁷ to about 10⁸. A population of adult salivary stem cells to be introduced into a recipient individual is generally at least about 75%, at least about 80%, at least about 85%, at least about 90%, at least about 95%, at least about 98%, or more than 98%, adult salivary stem cells.

The adult salivary stem cells to be introduced into the recipient individual may be referred to as a cell transplant. A cell transplant, as used herein, is the transplantation of one or more donor salivary stem cells into a recipient body, usually for the purpose of augmenting function of an organ or tissue in the recipient. The donor stem cells may originate from a salivary gland of the recipient, in which case the donor and the recipient are the same individual. In other aspects, the recipient is an individual to whom tissue or cells from another individual (donor), commonly of the same species, has been transferred. When the donor and recipient are not the same individual, the HLA antigens (or MHC antigens), which may be Class I or Class II, generally will be matched, although one or more of the HLA antigens may be different in the donor as compared to the recipient. The graft recipient and donor are generally mammals, e.g., humans. Laboratory animals, such as rodents, e.g. mice, rats, etc. are of interest. The cells may be allogeneic, autologous, or xenogeneic with respect to the recipient.

The cells may be provided as a suspension, which suspension includes one or more survival factors. As used herein, the term “survival factors” refers to biologically active agents that are provided in a formulation for the suspension of cells prior to transplantation. The presence of survival factor(s) enhances the survival of cells after the cells are transferred into the body of a recipient. Survival factors may be utilized as one or a cocktail of factors. In some embodiments, the survival factors are also utilized as culture additives for a period of time prior to transplantation.

The donor salivary stem cells may be administered in any physiologically acceptable excipient including an isotonic excipient prepared under sufficiently sterile conditions for human administration. For general principles in medicinal formulation, the reader is referred to Cell Therapy: Stem Cell Transplantation, Gene Therapy, and Cellular Immunotherapy, by G. Morstyn & W. Sheridan eds, Cambridge University Press, 1996; and Hematopoietic Stem Cell Therapy, E. D. Ball, J. Lister & P. Law, Churchill Livingstone, 2000. Choice of the cellular excipient and any accompanying elements of the composition will be adapted in accordance with the route and device used for administration. The cells may be introduced by injection, catheter, or the like. The cells may be frozen at liquid nitrogen temperatures and stored for long periods of time, being capable of use on thawing. If frozen, the cells may be stored, e.g., in a 10% dimethylsulfoxide (DMSO), 50% fetal calf serum (FCS) (or other suitable serum or serum substitute), 40% RPMI 1640 medium (or other suitable culture medium).

The cell formulations may be used for tissue (e.g., salivary gland) reconstitution or regeneration in a human patient or other subject in need of such treatment, e.g., a recipient individual having head and neck cancer who has undergone radiation treatment for the cancer. The cells are administered in a manner that permits them to graft or migrate to the intended tissue site and reconstitute or regenerate the functionally deficient area (e.g., an irradiated salivary gland).

The salivary stem cells may also be genetically modified to enhance survival, control proliferation, and the like. Cells may be genetically altering by transfection or transduction with a suitable vector, homologous recombination, or other appropriate technique, so that they express a gene of interest. For example, cells can be transfected with genes encoding a telomerase catalytic component (TERT), e.g., under a heterologous promoter that increases telomerase expression beyond what occurs under the endogenous promoter, (see International Patent Application WO 98/14592). In other embodiments, a selectable marker is introduced, to provide for greater purity of the desired differentiating cell. Cells may be genetically altered using vector containing supernatants over an 8-16 h period, and then exchanged into growth medium for 1-2 days. Genetically altered cells are selected using a drug selection agent such as puromycin, G418, or blasticidin, and then recultured.

### Methods of Treating Dry Eye Disease

The present invention also includes methods for treating or preventing dry eye disease. As used herein, “dry eye disease” (also known as “dry eye syndrome,” “dry eye disorder,” etc.) refers to any disease, condition or affliction characterized by one or more of: (a) decrease in tear production; (b) increase in tear film evaporation; (c) loss of mucous-containing conjunctival goblet cells; (d) desquamation of the corneal epithelium; and/or (e) destabilization of the cornea-tear interface. Dry eye disease may be characterized, according to known clinical criteria, as mild, moderate, moderate-to-severe, and severe dry eye disease. Accordingly, the present invention provides methods of treating any degree of dry eye disease, including mild dry eye disease, moderate dry eye disease, moderate-to-severe dry eye disease, or severe dry eye disease. Dry eye disease may be acute or chronic. Accordingly, the present invention provides methods of treating either acute or chronic dry eye disease. Dry eye disease may also be categorized as either “tear deficient dry eye disease” or “evaporative dry eye disease.” Accordingly, the present invention provides methods of treating tear deficient dry eye disease and/or evaporative dry eye disease.

According to the present invention, “dry eye disease” includes, e.g., age-related dry eye, blepharitis, conjunctivitis, corneal desquamation, corneal infiltrates, epithelial edema, giant papillary conjunctivitis, hypoxia, keratoconjunctivitis sicca (KCS), microbial keratitis, microcysts, ocular cicatrical pemphigoid, Stevens-Johnson syndrome, Sjogren's syndrome, and ulcerative keratitis. “Dry eye disease” also includes dry eye conditions associated with corneal injury, corneal surgery (including LASIK), radiation therapy, contact lens usage, infection, nutritional disorders or deficiencies, pharmacologic agents, eye stress, glandular and tissue destruction, exposure to pollutants and environmental conditions (e.g., smog, smoke, excessively dry air), airborne particulates, autoimmune and other immunodeficient disorders, and other conditions that impair or inhibit the ability of an individual to blink The methods of the present invention may be used to treat or prevent any of the foregoing conditions that fall under the definition of “dry eye disease”.

The methods of treating dry eye include administering a therapeutically effective amount of at least one isolated monoterpene to an individual in need thereof.

### Monoterpenes

A subject method involves use of compounds that are monoterpenes. In some cases, the monoterpenes are isolated. In some embodiments, the monoterpene is an ALDH3 agonist.

In some embodiments, a suitable monoterpene selectively modulates (e.g., increases) an enzymatic activity of ALDH3 (also referred to herein as ALDH3A1). For example, in some embodiments, a suitable isolated monoterpene increases an enzymatic activity of ALDH3, but does not substantially increase the same enzymatic activity of an ALDH isozyme other than ALDH3, e.g., the monoterpene increases an enzymatic activity of an ALDH isozyme other than ALDH3, if at all, by no more than about 15%, e.g., by less than 15%, less than 10%, less than 5%, or less than 1%.

A suitable monoterpene increases an enzymatic activity of an ALDH3 polypeptide by at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 100% (or two-fold), at least about 2.5-fold, at least about 5-fold, at least about 10-fold, at least about 15-fold, at least about 20-fold, at least about 25-fold, or at least about 50-fold, or greater than 50-fold, when compared to the enzymatic activity of the ALDH3 polypeptide in the absence of the monoterpene.

In some embodiments, a suitable monoterpene increases an enzymatic activity (e.g., an aldehyde dehydrogenase activity, a reductase activity, or an esterase activity) of an ALDH3 polypeptide, by at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 100% (or two-fold), at least about 2.5-fold, at least about 5-fold, at least about 10-fold, at least about 15-fold, at least about 20-fold, at least about 25-fold, or at least about 50-fold, or greater than 50-fold, when compared to the enzymatic activity of the ALDH3 polypeptide in the absence of the agonist.

In some embodiments, a suitable monoterpene has an EC₅₀ (half maximal effective concentration) of from about 1 nM to about 1 mM, e.g., from about 1 nM to about 10 nM, from about 10 nM to about 15 nM, from about 15 nM to about 25 nM, from about 25 nM to about 50 nM, from about 50 nM to about 75 nM, from about 75 nM to about 100 nM, from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 450 nM, from about 450 nM to about 500 nM, from about 500 nM to about 750 nM, from about 750 nM to about 1 μM, from about 1 μM to about 10 μM, from about 10 μM to about 25 μM, from about 25 μM to about 50 μM, from about 50 μM to about 75 μM, from about 75 μM to about 100 μM, from about 100 μM to about 250 μM, from about 250 μM to about 500 μM, or from about 500 μM to about 1 mM.

Whether a monoterpene compound is an ALDH agonist can be readily ascertained. Assays for dehydrogenase activity of ALDH are known in the art, and any known assay can be used. Examples of dehydrogenase assays are found in various publications, including, e.g., Sheikh et al. ((1997) J. Biol. Chem. 272:18817-18822); Vallari and Pietruszko (1984) J. Biol. Chem. 259:4922; and Farres et al. ((1994) J. Biol. Chem. 269:13854-13860).

As an example of an assay for dehydrogenase activity, ALDH aldehyde dehydrogenase activity is assayed at 25° C. in 50 mM sodium pyrophosphate HCl buffer, pH 9.0, 100 mM sodium phosphate buffer, pH 7.4, or 50 mM sodium phosphate buffer, pH 7.4, where the buffer includes NAD⁺ (e.g., 0.8 mM NAD⁺, or higher, e.g., 1 mM, 2 mM, or 5 mM NAD⁺) and an aldehyde substrate such as 14 μM propionaldehyde. Reduction of NAD⁺ is monitored at 340 nm using a spectrophotometer, or by fluorescence increase using a fluoromicrophotometer. Enzymatic activity can be assayed using a standard spectrophotometric method, e.g., by measuring a reductive reaction of the oxidized form of nicotinamide adenine dinucleotide (NAD⁺) to its reduced form, NADH, at 340 nm, as described in US 2005/0171043; and WO 2005/057213. In an exemplary assay, the reaction is carried out at 25° C. in 0.1 sodium pyrophosphate (NaPPi) buffer, pH 9.0, 2.4 mM NAD⁺ and 10 mM acetaldehyde as the substrate. Enzymatic activity is measured by a reductive reaction of NAD⁺ to NADH at 340 nm, as described in US 2005/0171043; and WO 2005/057213. Alternatively, the production of NADH can be coupled with another enzymatic reaction that consumes NADH and that provides for a detectable signal. An example of such an enzymatic reaction is a diaphorase-based reaction, which reduces resazurin to its oxidized fluorescent compound resorufin, as described in US 2005/0171043; and WO 2005/057213. Detection of fluorescent resorufin at 590 nm provides amplified and more sensitive signals for any change in ALDH aldehyde dehydrogenase enzymatic activity. NADP⁺ can be used in place of NAD⁺ in this assay. Suitable substrates include, but are not limited to, octylaldehyde, phenylacetaldehyde, retinaldehyde, and 4-hydroxynonenal.

As another example, the effect of a monoterpene compound on aldehyde dehydrogenase activity of an ALDH polypeptide can be assayed as described in Wierzchowski et al. ((1996) Analytica Chimica Acta 319:209), in which a fluorogenic synthetic substrate, e.g., 7-methoxy-1-naphthaldehyde is used. For example, the reaction could include 7-methoxy-1-naphthaldehyde, NAD⁺, an ALDH polypeptide, and a monoterpene to be tested; fluorescence (excitation, 330 nm; emission 390 nm) is measured as a readout of enzymatic activity.

Whether a monoterpene compound increases an esterase activity of an ALDH polypeptide can be determined using any known assay for esterase activity. For example, esterase activity of ALDH can be determined by monitoring the rate of p-nitrophenol formation at 400 nm in 25 mM N,N-Bis (2-hydroxyethyl)-2-amino ethanesulfonic acid (BES) (pH 7.5) with 800 μM p-nitrophenyl acetate as the substrate at room temperature in the absence or presence of added NAD⁺. A pH-dependent molar extinction coefficient of 16 mM⁻¹cm⁻¹ at 400 nm for nitrophenol can be used. See, e.g., Larson et al. (2007) J. Biol. Chem. 282:12940). Esterase activity of an ALDH polypeptide can be determined by measuring the rate of p-nitrophenol formation at 400 nm in 50 mM Pipes (pH 7.4) with 1 mM p-nitrophenylacetate as the substrate. A molar extinction coefficient of 18.3×10³ M⁻¹cm⁻¹ at 400 nm for p-nitrophenolate can be used for calculating its rate of formation. See, e.g., Ho et al. (2005) Biochemistry 44:8022).

Whether a compound increases a reductase activity of ALDH can be determined using any known assay for reductase activity. A reductase activity of ALDH can be determined by measuring the rate of 1,2-glyceryl dinitrate and 1,3-glyceryl dinitrate formation using a thin layer chromatography (TLC) or liquid scintillation spectrometry method, using a radioactively labeled substrate. For example, 0.1 mM or 1 mM GTN (glyceryl trinitrate) is incubated with the assay mixture (1 ml) containing 100 mM KPi (pH 7.5), 0.5 mM EDTA, 1 mM NADH, 1 mM NADPH in the presence the ALDH. After incubation at 37° C. for about 10 minutes to about 30 minutes, the reaction is stopped and GTN and its metabolites are extracted with 3×4 ml ether and pooled, and the solvent is evaporated by a stream of nitrogen. The final volume is kept to less than 100 μl in ethanol for subsequent TLC separation and scintillation counting. See, e.g., Zhang and Stamler (2002) Proc. Natl. Acad. Sci. USA 99:8306.

In some embodiments, a suitable monoterpene is a compound selected from the group consisting of, (R)-(+)-limonene, (S)-(−)-limonene, alpha-pinene, beta-pinene, alpha-terpinene, gamma-terpinene, terpineol, alpha-phellandrene, beta phellandrene, (S)-(−)-perillyl alcohol and (R)-(+)-perillyl alcohol or a pharmaceutically acceptable salt thereof. In certain cases, the monoterpene is (R)-(+)-limonene or a pharmaceutically acceptable salt thereof.

In some embodiments, a suitable monoterpene is isolated and is pure, e.g., at least 80%, at least about 90% pure, at least about 98% pure, or at least about 99% pure, by weight.

The present disclosure provides for use of monoterpene compounds isolated from natural extracts, e.g., extracts of plants and other organisms that naturally contain monoterpene compounds. Natural formulations and extracts can comprise at least one monoterpene in an amount by weight of from about 0.01% to about 30%, or from about 30% to about 80%, e.g., from about 0.01% to about 0.05%, from about 0.05% to about 0.1%, from about 0.1% to about 0.5%, from about 0.5% to about 1%, from about 1% to about 2.5%, from about 2.5% to about 5%, from about 5% to about 7.5%, from about 7.5% to about 10%, from about 10% to about 12.5%, from about 12.5% to about 15%, from about 15% to about 20%, from about 20% to about 25%, or from about 25% to about 30%. In some embodiments, a suitable natural formulation or natural extract comprises at least one monoterpene in an amount by weight of from about 30% to about 35%, from about 35% to about 40%, from about 40% to about 45%, from about 45% to about 50%, from about 50% to about 60%, from about 60% to about 70%, or from about 70% to about 80%. As used herein, a “natural formulation” or a “natural extract” can include components of a plant or other natural source of monoterpenes but does not exclude inclusion of components not normally found in a plant source of monoterpenes, e.g., the “natural formulation” or “natural extract” can include added components not normally found in a plant source.

In certain cases, the monoterpene is an isolated extract, such as a traditional Chinese medicine extract. In some embodiments the monoterpene has a structure according to any of Alda-341 to Alda-347, Alda-351 and Alda-364 shown below.

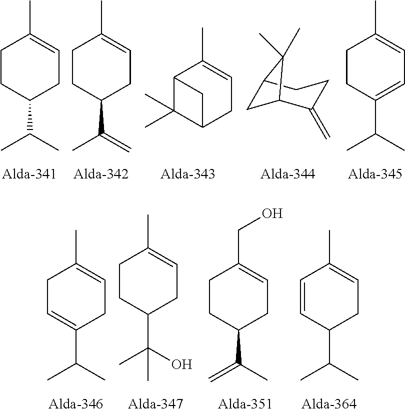

A plant or plant part can be extracted either singly or sequentially with one or more of an aqueous solution, an alcohol, a polar organic solvent, and a non-polar organic solvent. In some embodiments, the monoterpene is hydrophobic and is present in an organic phase of a natural extract. For example, a plant or a plant part can be extracted with an organic solvent such as hexane, ethyl acetate, methylene chloride or chloroform. In some embodiments, the plant or plant part is extracted with alcohol, e.g., methanol or butanol. In some embodiments, the plant or plant part is extracted with methanol:hexane (1:1 vol:vol). In some embodiments, the plant or plant part is extracted with methanol:hexane from 95:5 to 1:1. In some embodiments, the plant or plant part is extracted sequentially with an alcohol, then with an alcohol:hexane mixture. Polar organic solvents include, e.g., tetrahydrofuran, acetonitrile, acetone, and isopropyl alcohol. In some embodiments, the plant or plant part is extracted with a polar organic solvent. In some embodiments, the plant or plant part is extracted with methanol or hexane.

The natural extract can be obtained by extracting a plant or plant part at a temperature of from about 15° C. to about 20° C., from about 20° C. to about 25° C., from about 25° C. to about 30° C., from about 30° C. to about 35° C., from about 35° C. to about 40° C., from about 40° C. to about 45° C., from about 45° C. to about 50° C., from about 50° C. to about 60° C., from about 60° C. to about 70° C., from about 70° C. to about 80° C., from about 80° C. to about 90° C., or from about 90° C. to about 100° C.

A natural extract includes an extract of a whole plant or one or more parts of a plant, where plant parts include leaves, stems, rhizomes, roots, tubers, bulbs, flowers, bark, seeds, fruit, and the like. Thus, sources of the monoterpenes include, e.g., whole plant or one or more parts of a plant, where plant parts include leaves, stems, rhizomes, tubers, bulbs, roots, flowers, bark, seeds, fruit, and the like. Prior to extraction, the plant or plant part can be subjected to one or more processing steps; e.g., prior to extraction, the plant or plant part can be dried, powdered, frozen, steamed, ground, pulverized, or fermented. Pulverizing can be achieved by carrying out one or more of homogenizing, milling, grinding, chopping, blending, cutting, and tearing.

Combinations of two or more extracts are also contemplated, e.g., extracts of two or more different plant parts from the same plant; extracts from two or more plants of the same genus, where the plants are of two or more different species; extracts from two or more plants of two or more different genuses; a combination of an aqueous extract and an alcohol extract; a combination of an aqueous extract and a polar organic solvent extract; a combination of an aqueous extract and a non-polar organic solvent extract; etc.

A suitable natural extract can be formulated in any form convenient for use, e.g., a lozenge, a capsule, a powder, a liquid solution, a gel, etc. Any of a variety of components can be added to a natural extract, including, e.g., fillers, binders, sweeteners, flavors and other ingredients. Nearly any excipients that are known for use in the preparation of oral dosage pharmaceutical products, or natural supplement products, can be used. Examples of such excipients include without limitation, carbomer, carboxymethylcellulose sodium, cellulose, dextrin, dextrose, ethylcellulose, fructose, gelatin, guar gum, hydroxyethyl cellulose, hydroxypropyl cellulose, hydroxypropyl methylcellulose, glucose, maltodextrin, mannitol, methylcellulose, microcrystalline cellulose, polymethacrylates, povidone, sorbitol, starches, sucrose, sugar, sucralose, stevia, and flavor agents.

### Pharmaceutical Compositions, Dosages, Routes of Administration

In some instances, as discussed above, a monoterpene (e.g., an ALDH3 agonist) can be used to increase the number of adult salivary stem cells in vivo, e.g., an effective amount of an ALDH agonist is administered to an individual in need thereof. In some instances, a monoterpene can be used to protect adult salivary stem cells in vivo. In some instances, a monoterpene can be used to improve salivary gland function in vivo. The terms “monoterpene” and “ALDH3 agonist” are also referred to herein as “active agent.” For administration to an individual, a suitable monoterpene is formulated with one or more pharmaceutically acceptable excipients. A wide variety of pharmaceutically acceptable excipients are known in the art and need not be discussed in detail herein. Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds., 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

The term “unit dosage form,” as used herein, refers to physically discrete units suitable as unitary dosages for human and animal subjects, each unit containing a predetermined quantity of an active agent (e.g., a monoterpene) calculated in an amount sufficient to produce the desired effect in association with a pharmaceutically acceptable diluent, carrier or vehicle. The specifications for an active agent depend on the particular compound employed and the effect to be achieved, and the pharmacodynamics associated with each compound in the host.

In the subject methods, a suitable monoterpene may be administered to the host using any convenient means capable of resulting in the desired outcome, e.g., treatment of dry mouth and dry eye, etc. Thus, a suitable monoterpene can be incorporated into a variety of formulations for therapeutic administration. More particularly, a suitable monoterpene can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols.

Suitable excipient vehicles are, for example, water, saline, dextrose, glycerol, ethanol, or the like, and combinations thereof. In addition, if desired, the vehicle may contain minor amounts of auxiliary substances such as wetting or emulsifying agents or pH buffering agents. Actual methods of preparing such dosage forms are known, or will be apparent, to those skilled in the art. See, e.g., Remington's Pharmaceutical Sciences, Mack Publishing Company, Easton, Pa., 17th edition, 1985. The composition or formulation to be administered will, in any event, contain a quantity of the agent adequate to achieve the desired state in the subject being treated.

In pharmaceutical dosage forms, a suitable monoterpene (“active agent”) may be administered in the form of its pharmaceutically acceptable salts, or an active agent may be used alone or in appropriate association, as well as in combination, with other pharmaceutically active compounds. The following methods and excipients are merely exemplary and are in no way limiting.

For oral preparations, an active agent can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

An active agent can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

An active agent can be utilized in aerosol formulation to be administered via inhalation. An active agent can be formulated into pressurized acceptable propellants such as dichlorodifluoromethane, propane, nitrogen and the like.

Furthermore, an active agent can be made into suppositories by mixing with a variety of bases such as emulsifying bases or water-soluble bases. An active agent can be administered rectally via a suppository. The suppository can include vehicles such as cocoa butter, carbowaxes and polyethylene glycol monomethyl ethers, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the active agent. Similarly, unit dosage forms for injection or intravenous administration may comprise an active agent in a composition as a solution in sterile water, normal saline or another pharmaceutically acceptable carrier.

An active agent can be formulated for administration by injection. Typically, injectable compositions are prepared as liquid solutions or suspensions; solid forms suitable for solution in, or suspension in, liquid vehicles prior to injection may also be prepared. The preparation may also be emulsified, or the active ingredient encapsulated in liposome vehicles.

In certain embodiments, the active agent can be formulated for administration by ocular, intraocular, intravitreal or subconjunctival injection. In other embodiments, the active agent can be formulated for administration by topical administration, e.g., via eye drops or other liquid, gel, ointment or fluid which contains the monoterpene and can be applied directly to the eye. Other formulations can be configured for administration of the monoterpene via depots placed on or around the eye, including active agent embedded in a contact lens or other ocular surface adherent device, or in a punctual plug.

### Dosages and Dosing

Depending on the subject and condition being treated and on the administration route, an active agent may be administered in dosages of, for example, 0.1 μg to 500 mg/kg body weight per day, e.g., from about 0.1 μg/kg body weight per day to about 1 μg/kg body weight per day, from about 1 μg/kg body weight per day to about 25 μg/kg body weight per day, from about 25 μg/kg body weight per day to about 50 μg/kg body weight per day, from about 50 μg/kg body weight per day to about 100 μg/kg body weight per day, from about 100 μg/kg body weight per day to about 500 μg/kg body weight per day, from about 500 μg/kg body weight per day to about 1 mg/kg body weight per day, from about 1 mg/kg body weight per day to about 25 mg/kg body weight per day, from about 25 mg/kg body weight per day to about 50 mg/kg body weight per day, from about 50 mg/kg body weight per day to about 100 mg/kg body weight per day, from about 100 mg/kg body weight per day to about 250 mg/kg body weight per day, or from about 250 mg/kg body weight per day to about 500 mg/kg body weight per day. The range is broad, since in general the efficacy of a therapeutic effect for different mammals varies widely with doses typically being 20, 30 or even 40 times smaller (per unit body weight) in man than in the rat. Similarly, the mode of administration can have a large effect on dosage. Thus, for example, oral dosages may be about ten times the injection dose. Higher doses may be used for localized routes of delivery.

For example, a monoterpene can be administered in an amount of from about 1 mg to about 1000 mg per dose, e.g., from about 1 mg to about 5 mg, from about 5 mg to about 10 mg, from about 10 mg to about 20 mg, from about 20 mg to about 25 mg, from about 25 mg to about 50 mg, from about 50 mg to about 75 mg, from about 75 mg to about 100 mg, from about 100 mg to about 125 mg, from about 125 mg to about 150 mg, from about 150 mg to about 175 mg, from about 175 mg to about 200 mg, from about 200 mg to about 225 mg, from about 225 mg to about 250 mg, from about 250 mg to about 300 mg, from about 300 mg to about 350 mg, from about 350 mg to about 400 mg, from about 400 mg to about 450 mg, from about 450 mg to about 500 mg, from about 500 mg to about 750 mg, or from about 750 mg to about 1000 mg per dose.

An exemplary dosage may be a solution suitable for intravenous administration; a tablet taken from two to six times daily, or one time-release capsule or tablet taken once a day and containing a proportionally higher content of active ingredient, etc. The time-release effect may be obtained by capsule materials that dissolve at different pH values, by capsules that release slowly by osmotic pressure, or by any other known means of controlled release.

Those of skill in the art will readily appreciate that dose levels can vary as a function of the specific compound, the severity of the symptoms and the susceptibility of the subject to side effects. Preferred dosages for a given compound are readily determinable by those of skill in the art by a variety of means.

Although the dosage used will vary depending on the clinical goals to be achieved, a suitable dosage range is in some embodiments one which provides up to about 1 μg to about 1,000 μg or about 10,000 μg of an active agent in a blood sample taken from the individual being treated, about 24 hours after administration of the compound to the individual.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more compounds of the invention. Similarly, unit dosage forms for injection or intravenous administration may comprise the compound(s) in a composition as a solution in sterile water, normal saline or another pharmaceutically acceptable carrier.

In some embodiments, multiple doses of an active agent are administered. The frequency of administration of a compound (“active agent”) can vary depending on any of a variety of factors, e.g., severity of the symptoms, etc. For example, in some embodiments, an active agent is administered once per month, twice per month, three times per month, every other week (qow), once per week (qw), twice per week (biw), three times per week (tiw), four times per week, five times per week, six times per week, every other day (qod), daily (qd), twice a day (bid), or three times a day (tid). As discussed above, in some embodiments, an active agent is administered continuously.

The duration of administration of an active agent, e.g., the period of time over which an active agent is administered, can vary, depending on any of a variety of factors, e.g., patient response, etc. For example, an active agent can be administered over a period of time ranging from about one day to about one week, from about two weeks to about four weeks, from about one month to about two months, or from about two months to about four months, or more.

### Routes of Administration

A suitable monoterpene is administered to an individual using any available method and route suitable for drug delivery, including in vivo and ex vivo methods, as well as systemic and localized routes of administration. Administration can be acute (e.g., of short duration, e.g., a single administration, administration for one day to one week), or chronic (e.g., of long duration, e.g., administration for longer than one week, e.g., administration over a period of time of from about 2 weeks to about one month, from about one month to about 3 months, from about 3 months to about 6 months, or more).

Conventional and pharmaceutically acceptable routes of administration include intranasal, intramuscular, intratracheal, subcutaneous, intradermal, transdermal, sublingual, topical application, intravenous, ocular (e.g., topically to the eye, intravitreal, etc.), rectal, nasal, oral, and other enteral and parenteral routes of administration. Routes of administration may be combined, if desired, or adjusted depending upon the agent and/or the desired effect. The compound can be administered in a single dose or in multiple doses.

An active agent can be administered to a host using any available conventional methods and routes suitable for delivery of conventional drugs, including systemic or localized routes. In general, routes of administration contemplated by the invention include, but are not necessarily limited to, enteral, parenteral, and inhalational routes.

Parenteral routes of administration other than inhalation administration include, but are not necessarily limited to, topical, transdermal, subcutaneous, intramuscular, intraorbital, intracapsular, intraspinal, intrasternal, ocular, and intravenous routes, i.e., any route of administration other than through the alimentary canal. Parenteral administration can be carried to effect systemic or local delivery of the agent. Where systemic delivery is desired, administration typically involves invasive or systemically absorbed topical or mucosal administration of pharmaceutical preparations.

The agent can also be delivered to the subject by enteral administration. Enteral routes of administration include, but are not necessarily limited to, oral and rectal (e.g., using a suppository) delivery.

Methods of administration of a suitable monoterpene through the skin or mucosa include, but are not necessarily limited to, topical application of a suitable pharmaceutical preparation, transdermal transmission, injection and epidermal administration. For transdermal transmission, absorption promoters or iontophoresis are suitable methods. Iontophoretic transmission may be accomplished using commercially available “patches” which deliver their product continuously via electric pulses through unbroken skin for periods of several days or more.

### Treatment Methods

The present disclosure provides various treatment methods, generally involving administering to an individual in need thereof an effective amount of at least one isolated monoterpene and/or a treated population of adult salivary stem cells (e.g., salivary stem cells expanded in vitro or ex vivo by contacting the salivary stem cells with at least one isolated monoterpene) and in some cases, subjecting the individual to radiation treatment before or after administration of the monoterpene.

Local and/or Systemic Monoterpene Administration

As noted above, individuals with head and neck cancer commonly undergo radiotherapy (RT) which often results in permanent damage to the salivary glands, causing loss of function and subsequent RT-related xerostomia or dry mouth. Such treatment may also dry the mucous membranes in the eyes, resulting in dry eye disease. Treatment methods of the present disclosure may include in vivo activation of the ALDH enzyme ALDH3, in tissues of the head and neck region (one or more salivary glands, for example) of an individual with head and neck cancer who will undergo, or has undergone, radiotherapy to treat the head and neck cancer. The methods may include administering a monoterpene systemically (e.g., by oral, intravenous, or other systemic administration) or locally (e.g., by local injection and/or topical application at a target site of a composition that includes a monoterpene). According to one embodiment of the present disclosure, the monoterpene may be administered (e.g., systemically and/or locally) before the individual with head and neck cancer undergoes radiation therapy. In another embodiment, the ALDH agonist may be administered (e.g., systemically and/or locally) after the individual with head and neck cancer undergoes radiation therapy. In yet another embodiment, the monoterpene is administered before and after the individual undergoes radiation therapy. In certain embodiments, the monoterpene is administered continuously for a period of time before the individual is subjected to radiation therapy. In certain embodiments, the monoterpene is administered continuously for a period of time after the individual is subjected to radiation therapy. In some cases, the monoterpene is administered continuously for a period of time before and after the individual undergoes radiation therapy.

As noted above, in some embodiments, a monoterpene (e.g., an activator of ALDH3) is administered as a “pretreatment” to an individual before the individual undergoes radiation treatment, e.g., from about 1 hour to about 1 week before the radiation treatment, e.g., from about 1 hour to about 2 hours, from about 2 hours to about 4 hours, from about 4 hours to about 8 hours, from about 8 hours to about 12 hours, from about 12 hours to about 16 hours, from about 16 hours to about 24 hours, from about 24 hours to about 36 hours, from about 36 hours to about 48 hours, from about 48 hours to about 72 hours, or from about 72 hours to about 1 week preceding the radiation treatment. In some embodiments pretreatment with the monoterpene is continuous over the period of time preceding radiation treatment.

Pretreatment with a monoterpene (e.g., an activator of ALDH3) is useful, for example, to expand the number of adult salivary stem cells in vivo, such that the probability of a sufficient number of stem cells surviving the radiation treatment is increased. The above situation is only one example of a circumstance when a subject would benefit from pretreatment with a suitable monoterpene.

In some embodiments, a suitable monoterpene is administered after radiation therapy. For example, a suitable monoterpene administered after radiation treatment is effective for mitigating the adverse effects of the radiation treatment on the salivary gland and corresponding salivary function. In some embodiments, a suitable monoterpene is administered within 1 minute to within 15 hours, e.g., from about 1 minute to about 5 minutes, from about 5 minutes to about 10 minutes, from about 10 minutes to about 15 minutes, from about 15 minutes to about 30 minutes, from about 30 minutes to about 60 minutes, from about 60 minutes to about 2 hours, from about 2 hours to about 4 hours, from about 4 hours to about 8 hours, from about 8 hours to about 12 hours, or from about 12 hours to about 15 hours, following the ischemic event. In some embodiments, an increased concentration of monoterpene is maintained in the plasma for at least several hours to several days following the radiation treatment, e.g., from about 1 day to about 1 week, from about 1 week to about 2 weeks, from about 2 weeks to about 3 weeks, from about 4 weeks to about 5 weeks, from about 6 weeks to about 7 weeks, from about 7 weeks to about 8 weeks, from about 8 weeks to about 9 weeks, from about 9 weeks to about 10 weeks, from about 10 weeks to about 11 weeks, or from about 11 weeks to about 12 weeks, following the ischemic event.

For example, in some embodiments, a suitable monoterpene is administered to an individual with head and neck cancer within 1 minute to within 15 hours, e.g., from about 1 minute to about 5 minutes, from about 5 minutes to about 10 minutes, from about 10 minutes to about 15 minutes, from about 15 minutes to about 30 minutes, from about 30 minutes to about 60 minutes, from about 60 minutes to about 2 hours, from about 2 hours to about 4 hours, from about 4 hours to about 8 hours, from about 8 hours to about 12 hours, or from about 12 hours to about 15 hours, following radiation treatment.

As described above in the section entitled “Methods of Treating Adult Salivary Stem Cells”, the present disclosure provides methods that optionally include introducing a treated population of adult salivary stem cells (e.g., where one or more of expansion, protection and increased salivary gland function of the treated adult salivary stem cells is effected by contacting the cells with a monoterpene, e.g., an activator of ALDH3) into a recipient individual. Introduction of the treated salivary stem cells is useful in a variety of applications. For example, the introduced (e.g., “transplanted”) salivary stem cells may engraft to an irradiated salivary gland and differentiate into functional saliva-producing cells, thereby restoring or enhancing the function of the irradiated salivary gland.

In certain aspects, the present disclosure provides treatment regimens that combine the post-radiation therapy introduction of a treated population of adult salivary stem cells (e.g., as described above) with the pre- and/or post-radiotherapy administration (e.g., systemic and/or local administration) of monoterpene to an individual (e.g., as also described above). As such, the present disclosure provides a treatment regimen wherein an individual with head and neck cancer receives an administration of a monoterpene (e.g., an activator of ALDH3) before radiotherapy, the treatment regimen further including introducing into the individual a treated population of adult salivary stem cells as described above. The present disclosure further provides a treatment regimen wherein an individual with head and neck cancer receives an administration of a monoterpene (e.g., an activator of ALDH3) and an administration of a treated population of adult salivary stem cells, with both administrations occurring after the radiotherapy. As will be appreciated, the present disclosure also provides a treatment regimen in which a monoterpene is administered systemically and/or locally to an individual before and after radiotherapy, the treatment regimen further including the introduction of a treated population of adult salivary stem cells after the radiotherapy.

In certain aspects, the present disclosure provides treatment regimens that alleviate drying of the mucous membranes in the mouth and eyes of an individual who is undergoing radiation therapy for a head and neck cancer.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1

### To Determine if Loss of ALDH3 Impairs Normal Salivary Stem/Progenitor Cell (SSPC) Function and Accelerates Hyposalivation After Radiation

To determine the role of ALDH3A1 in SSPC self-renewal, salivary sphere formation was measured of SSPC-enriched EpCAM⁺CD24⁺ cells isolated by fluorescence-activated cell sorting (FACS) from SMG of C57BL/6 wild-type (WT) and Aldh3a1(−/−) mice.

Salisphere formation assay: Cells isolated from mouse submandibular glands (SMG) were FACS sorted using cell surface markers EpCAM and CD24 as previously described and seeded on matrigel (BD Biosciences, BD356235). Cells were grown in DMEM/F12+GlutaMax (Gibco, 10565-018) media containing 10% FBS, 1× anti-anti, 1% N2 supplement (Gibco), 20 ng/mL epidermal growth factor-2 (Sigma-Aldrich), 20 ng/mL fibroblast growth factor-2 (Sigma-Aldrich), 10 μg/mL insulin (Sigma-Aldrich), 1 μM dexamethasone (Sigma-Aldrich), 10 μM γ-27632 (Stem Cell Technologies). Spheres were passaged every 7 days by dissociating into single cells by incubating in 1 mg/mL dispase (BD Biosciences) for 20 minutes and 0.25% trypsin-EDTA for 20 minutes. Spheres were grown for 7 days and imaged with a BZ-X710 Keyence microscope using the z-stack function. Images were stitched together into a single image and analyzed using ImageJ (NIH) to quantify sphere number and area. Experiments were repeated at least 3 independent times.

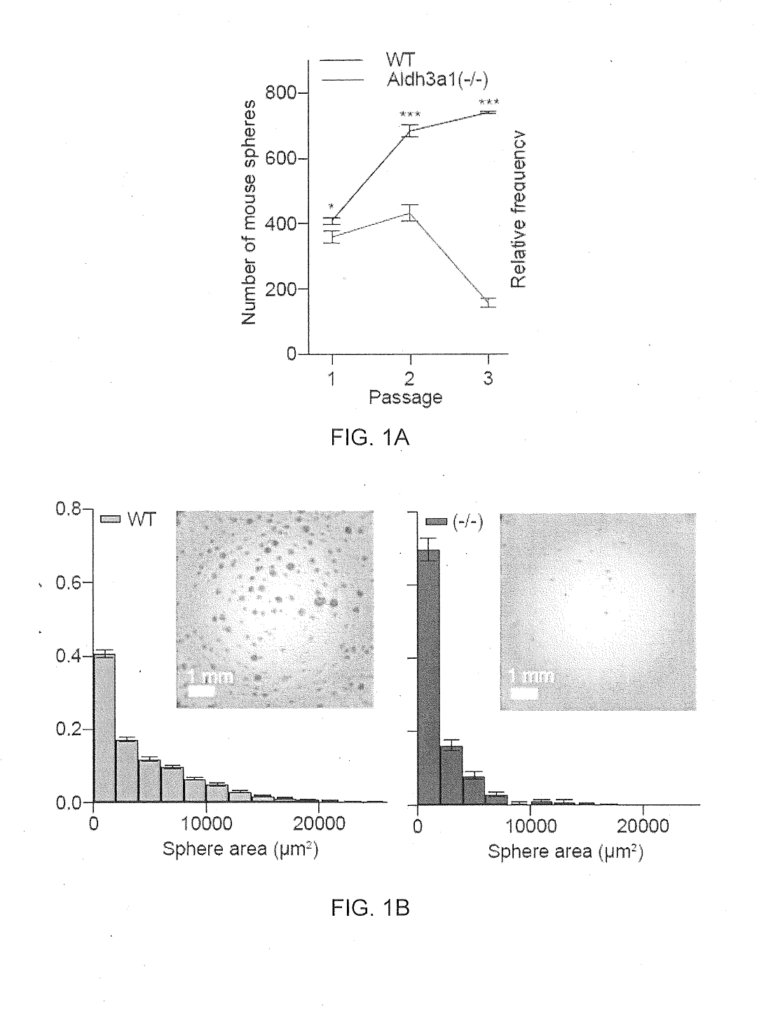

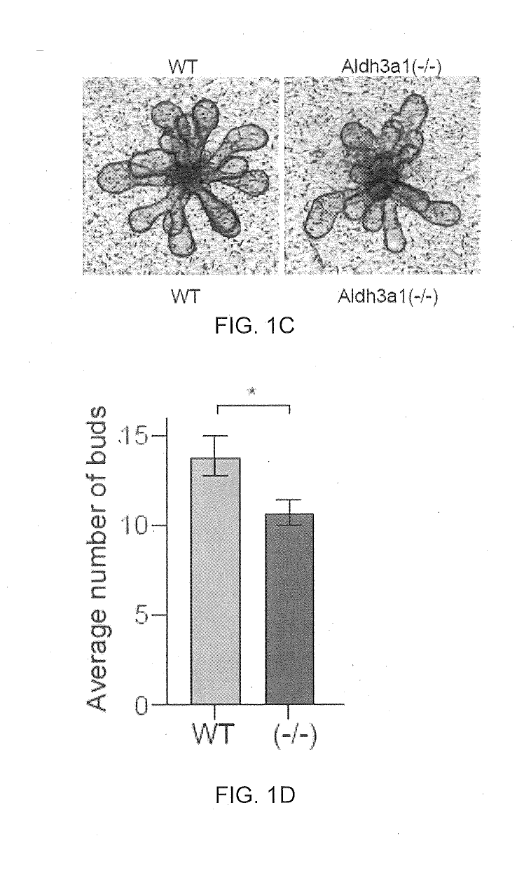

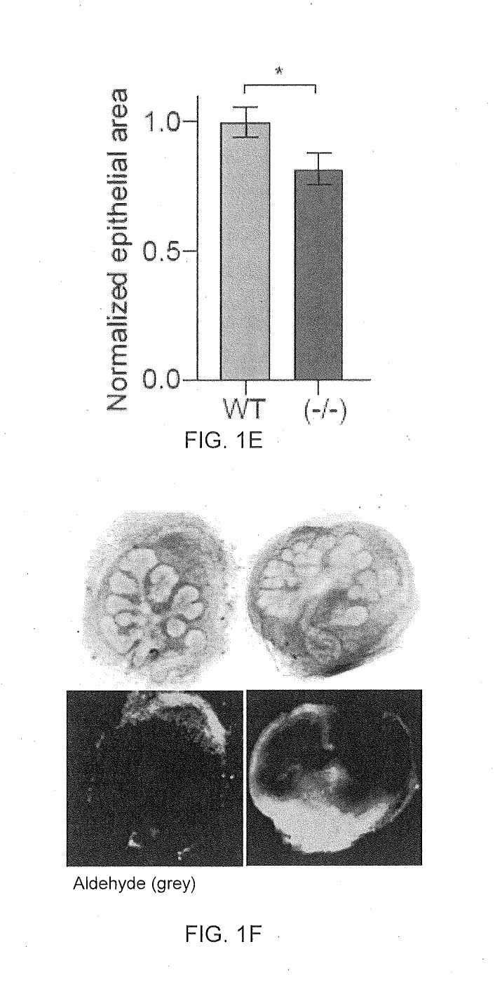

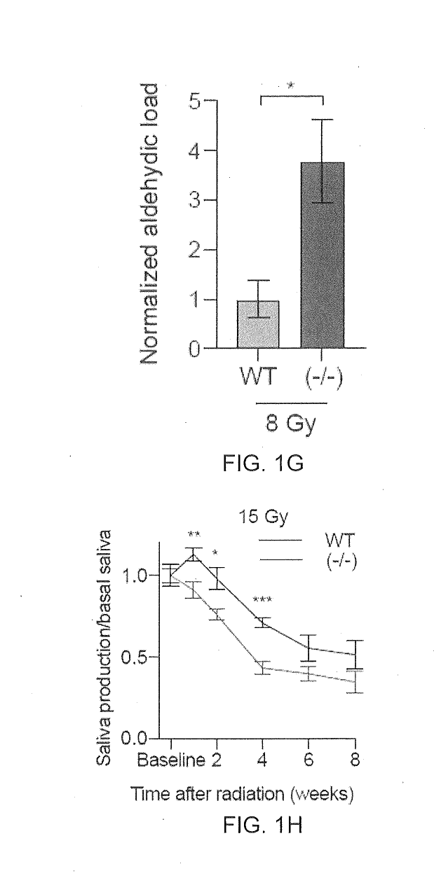

Compared with EpCAM⁺CD24⁺ cells from WT mice, EpCAM⁺CD24⁺ cells from Aldh3a1(−/−) mice demonstrated a progressive decrease in sphere forming ability over first, second, and third passages (FIG. 1, panel A). By the third passage, the number of salispheres derived from Aldh3a1(−/−) mice was approximately 80% lower than controls and were much smaller in size (measured as area) compared with those from WT mice (FIG. 1, panel B).

To further determine whether ALDH3A1 is necessary for normal SSPC function, isolated SMG epithelia dissected from embryonic day 13.5 (E13.5 mice) were used as a model due to their abundance of progenitor cells and substantial regenerative potential over short time periods (days as opposed to weeks for adult tissue).

Embryonic epithelial rudiment cultures: Epithelia and mesenchyme were separated using dispase treatment and mechanical dissection and cultured in a drop of laminin on a nucleopore filter over serum-free DMEM/F12 containing holotransferrin and ascorbic acid (complete media) as described for the E13 submandibular gland (Steinberg, Z., Myers, C., Heim, V. M., Lathrop, C. A., Rebustini, I. T., Stewart, J. S., Larsen, M., and Hoffman, M. P. (2005) FGFR2b signaling regulates ex vivo submandibular gland epithelial cell proliferation and branching morphogenesis. Development (Cambridge, England) 132, 1223-1234). Epithelia were cultured with 400 ng/ml FGF10 (R&D Systems) and 0.5 μl/ml heparin sulfate (Sigma Aldrich) in the presence or absence of 25-200 μM ALDH341 or vehicle (PEG) and were subjected to RNA isolation or fixed for immunostaining after 24-48 h.

Compared with WT C57BL/6, epithelia from Aldh3a1 (−/−) embryos demonstrated reduced formation of end buds—the regions from which acinar cells form—and reduced epithelial area after 24 h in culture, suggesting that ALDH3A1 plays a role in epithelial expansion (FIG. 1C).

To determine whether ALDH3A1 is required for the protection of SSPC and salivary function from radiation injury, it was first determined whether Aldh3a1(−/−) E13.5 embryonic SMGs accumulated more aldehydes after radiation. E13.5 SMG were cultured for 24 h, irradiated with 8 Gy, and treated with a novel DarkZone dye developed by Eric Kool's lab at Stanford (Stanford, Calif.) that fluorescently labels intracellular aldehydes.

DarkZone dye aldehyde assay in intact E13.5 embryonic SMG: E13.5 embryonic whole SMG were manually dissected and cultured in DMEM/F12 with 50 ug/mL transferrin and 50 ug/mL Vitamin C for 24 h. Glands were irradiated with 4 or 8 Gy and incubated with 20 uM DarkZone fluorescein aldehyde dye and 10 mM 2,4-Dimethoxyaniline catalyst (TCI America) for 1 h (Yuen, L. H., Saxena, N. S., Park, H. S., Weinberg, K., and Kool, E. T. (2016) Dark Hydrazone Fluorescence Labeling Agents Enable Imaging of Cellular Aldehydic Load. ACS chemical biology 11, 2312-2319). Glands were imaged 3 h after radiation using a Keyence BZ-X710 microscope GFP filter at 10× magnification. Average fluorescence intensity was quantified using Image J (NIH).

Three hours after radiation, Aldh3a1(−/−) embryonic SMG demonstrated more than 3-fold higher fluorescence intensity as compared to WT SMG, suggesting greater aldehydic accumulation (FIG. 1F-FIG. 1G).

Using a mouse model of radiation-induced hyposalivation, loss of salivary function after radiation in WT and Aldh3a1(−/−) mice was compared. Baseline pilocarpine-induced saliva production was collected for 15 minutes in 8-10 week old mice. SMG were locally irradiated with 30 Gy fractionated over 5 d (6 Gy/d) with the rest of the body lead-shielded. Pilocarpine-induced saliva production was measured at 1, 2, 4, 6, and 8 w after radiation (FIG. 1H). It was observed that Aldh3a1(−/−) mice experienced an accelerated decrease in saliva production after radiation compared with WT mice, suggesting that ALDH3A1 plays a protective role in SMG against radiation injury.

FIG. 1A shows EpCAM⁺CD24⁺ cells from C57BL/6 WT and Aldh(−/−) mice sorted by flow cytometry were seeded at a density of 10,000 cells per well and grown into spheres in 6 replicates per group. Average number of spheres per well was calculated by imaging each well and quantifying by Image J (NIH). Cells were passaged every 7 d for 3 passages.

FIG. 1B shows the area for WT (left) and Aldh(−/−) (right) spheres from passage 3 quantified by Image J (NIH) and represented as a histogram.

FIG. 1C shows representative SMG epithelia from C57BL/6 WT (left) and Aldh(−/−) (right) mouse embryos cultured for 24 h and imaged at 10× magnification.

FIG. 1D shows the bud number counted from 17 WT and 16 Aldh(−/−) epithelia.

FIG. 1E shows the average epithelial area quantified from 15 WT and 14 Aldh(−/−) epithelia using Image J (NIH) and normalized to WT.

FIG. 1F shows representative images of C57BL/6 WT (left) and Aldh(−/−) (right) E13.5 mouse embryonic SMG after 24 h in culture, incubated with DarkZone dye aldehyde sensor, and 3 h after 4 Gy taken at 10× magnification in brightfield (top) and with a GFP filter (bottom).

FIG. 1G shows quantification of fluorescence intensity representing aldehydic load by Image J (NIH) and normalized to WT.

FIG. 1H shows pilocarpine-induced saliva production collected for 15 minutes at baseline and after a single dose of 15 Gy to the SMG at 1, 2, 4, 6, and 8 weeks after radiation. Normalized to baseline saliva. N=10-11 mice/group. All error bars for this figure represent standard error of the mean (SEM). *=p<0.05; *=p<0.01; ***=p<0.001 (Student's t-test).

### Example 2

### Natural Product Screen to Indentify Small Molecule Activators of ALDH3

To determine if ALDH3A1 activation is sufficient to protect salivary glands from radiation-induced dysfunction, a library of 135 traditional Chinese medicine extracts donated from Sun Ten Pharmaceutical Co., Taiwan, was screened using a spectroscopic enzyme activity assay and purified recombinant ALDH3A1. At least seven extracts from this library showed an increase in ALDH3A1 activity. We then conducted high performance liquid chromatography (HPLC) fractionation of the extracts and nuclear magnetic resonance (NMR) characterization of the fractions. Based on the NMR fingerprint, we identified known constituents of similar fingerprints and purchased the commercially available compounds to test using the enzyme activity assay.

Methanol and hexane extractions of traditional Chinese medicine plants: 10 g of raw material was soaked in 100 mL of methanol or hexane overnight at 37° C. on a shaker. The mixture was passed through a qualitative filter paper (GE Healthcare Life Sciences Whatman, 270 mm diameter, grade 1). The remaining unfiltered material was soaked in 100 mL of methanol or hexane for an additional 2 hours. The second mixture was passed through a qualitative filter paper. The two filtrates were combined for a total volume of 200 mL and concentrated to 20 mL using a rotavapor (Buchi R-100). 1 mL was further concentrated to a powder using a vacuum.

HPLC: Hexane extract #139 was further extracted with a 1:2 mixture of water and acetonitrile and fractionated by reverse-phase HPLC (Agilent 1260 Infinity, C18 column, 250×4.6 mm). A linear gradient of water to acetonitrile (5% to 100%) over 30 minutes at a flow rate of 0.65 ml/min was used. Fractions were collected once per minute using an autosample collector. The displayed wavelength is 220 nm.

NMR: 1D ¹H NMR spectra were acquired at the Stanford Magnetic Resonance Laboratory on a Bruker Avance 500 MHz spectrometer (TopSpin v1.3) with sample temperature regulated to 25° C., 30° pulse, 16-264 scans, 1 sec pre-scan delay, 65536 total data points, 10330.58 Hz spectral width. Data were processed and analyzed using ACD Labs SpecHPLCtrus Processor.

Of the identified constituents, Alda-341, also known as D-limonene, had the lowest EC₅₀ of ˜14 μM and an Aₘₐₓ of ˜4.6.

ALDH enzymatic assay: ALDH1A1, ALDH1A2, 2, 3A1, 3A2, 4A1, 5A1, 7A1 were measured as previously described using 5 μg/ml of recombinant protein (Chen, C. H., Cruz, L. A., and Mochly-Rosen, D. (2015) Pharmacological recruitment of aldehyde dehydrogenase 3A1 (ALDH3A1) to assist ALDH2 in acetaldehyde and ethanol metabolism in vivo. Proceedings of the National Academy of Sciences of the United States of America 112, 3074-3079). Briefly, enzymatic activity was measured spectrophotometrically by the reduction of NAD⁺ to NADP⁺ at A₃₄₀ over 5 minutes in the presence of increasing concentrations of Alda-341 or DMSO vehicle control. Assays were conducted in 50 mM NaPPi buffer (pH 7.4) in the presence of 2.5 mM NAD⁺ and 10 mM substrate and measured in triplicate at 25° C. Dose response curve fits and EC₅₀'s were calculated using GraphPad Prism 7 software.

ALDH fluorescence-coupled enzymatic assay: ALDH enzyme activity was measured in cell lysate or using recombinant ALDH1A1, ALDH1A2, ALDH3A1 by the reduction of NAD⁺ to NADP⁺ amplified by the diaphorase conversation of resazurin to fluorescent resofurin (excitation 565 nm and emission 590 nm) over 5 minutes in the presence of Alda-341 or DMSO vehicle control. Assay conditions were modified from the original enzyme assay protocol above using 50 mM NaPPi buffer (pH 7.4), 2.5 mM NAD⁺, 10 mM acetaldehyde or all-trans-retinal substrate with the addition of 1 U/mL diaphorase and 0.1 mM resazurin for the secondary reaction. Measurements were collected in triplicate at 25° C.

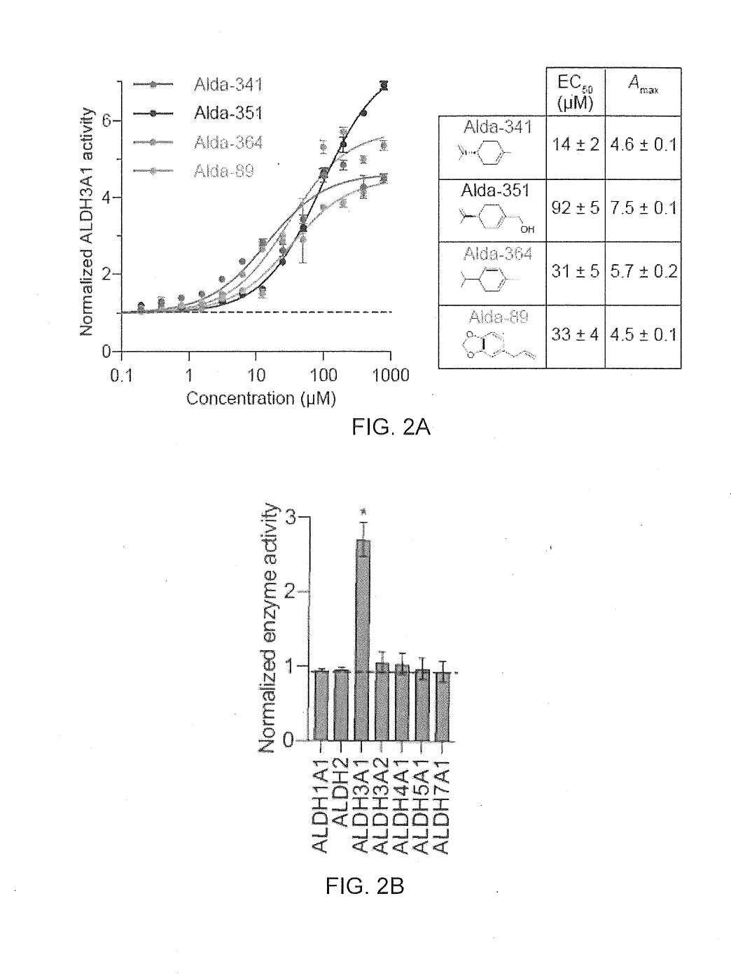

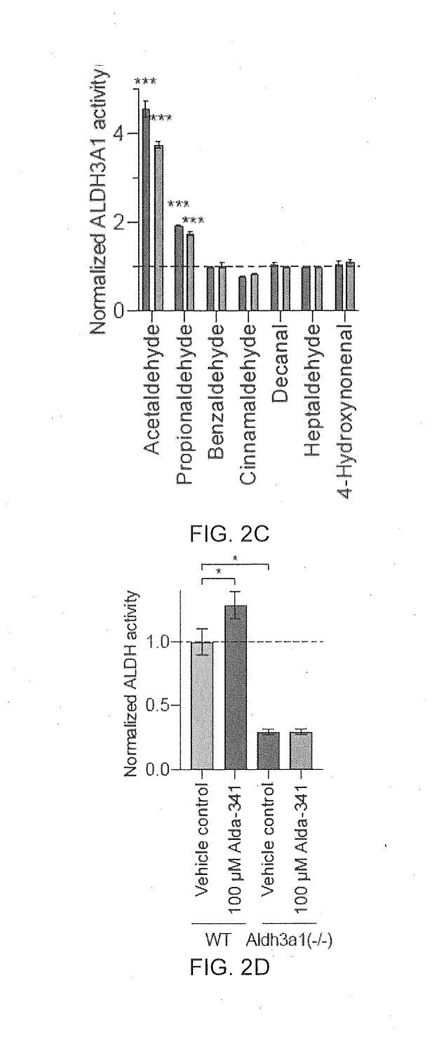

FIG. 2A shows dose response curves from the three top activators identified from the natural product screen along with safrole (Alda-89). The activity of both Alda-341 and Alda-89 appear to be substrate-specific; they only increase the catalytic activity of ALDH3A1 towards small aldehydes like acetaldehyde and propionaldehyde but not aromatic or long-chain aldehydes (FIG. 2B). Alda-341 did not increase the catalytic activity of ALDH1A1, ALDH2, ALDH3A2, ALDH4A1, ALDH5A1, or ALDH7A1 (FIG. 2C). Alda-341 also did not increase the catalytic activity of all trans-retinal in ALDH1A1, ALDH1A2, or ALDH3A1. Using a more sensitive fluorescence-coupled enzyme assay, it was observed that Aldh3a1(−/−) murine salivary sphere lysate demonstrated ˜30% of the enzymatic activity of WT lysate in the presence of acetaldehyde. Moreover, 100 μM of Alda-341 increased enzymatic activity of WT lysate by approximately 30% but did not increase the enzymatic activity of the lysate from Aldh3a1(−/−) mice (FIG. 2D).

FIG. 2A shows dose response curves from for three top activators identified from the natural product library screen using a spectrophotometric enzyme activity assay normalized to baseline activity. Activity measured in presence of compound concentrations from 6 nM to 400 μM. GraphPad Prism software used to calculate curve fit, EC₅₀ and Aₘₐₓ values, reported in table (right).

FIG. 2B shows enzyme activity which was measured using 5 μg/ml of recombinant ALDH isozymes, ALDH1A1, ALDH2, ALDH3A1, ALDH3A2, ALDH4A1, ALDH5A1, or ALDH7A1 in the presence of 10 mM acetaldehyde and 20 μM Alda-341.

FIG. 2C shows ALDH3A1 enzyme activity measured in the presence of 10 mM acetaldehyde, propionaldehyde, benzaldehyde, cinnamaldehyde, decanal, heptaldehyde, or 200 μM 4-hydroxynonenal and 100 μM Alda-341 or Alda-89.

FIG. 2D shows enzyme activity measured in 400 μg/mL of mouse primary salivary sphere lysate from C57BL/6 WT and Aldh3a1(−/−) mice using fluorescence-coupled enzymatic activity assay in the presence of 10 mM acetaldehyde and 100 μM Alda-341. All measurements were collected in triplicate, and error bars represent SEM. *=p<0.05; ***=p<0.001 (Student's t-test).

### Example 3

### To Determine if Activation of ALDH3 with Alda-341 Increases Normal SSPC Formation

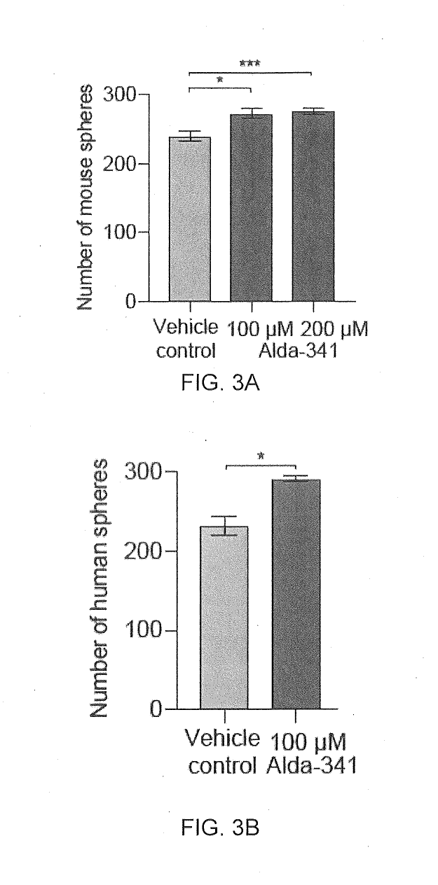

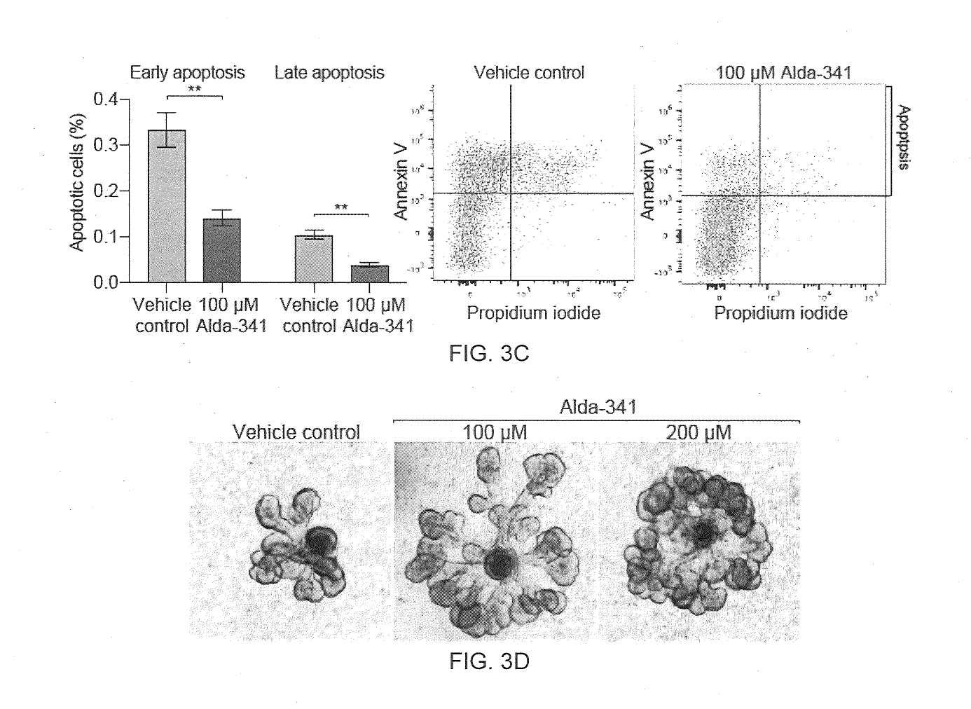

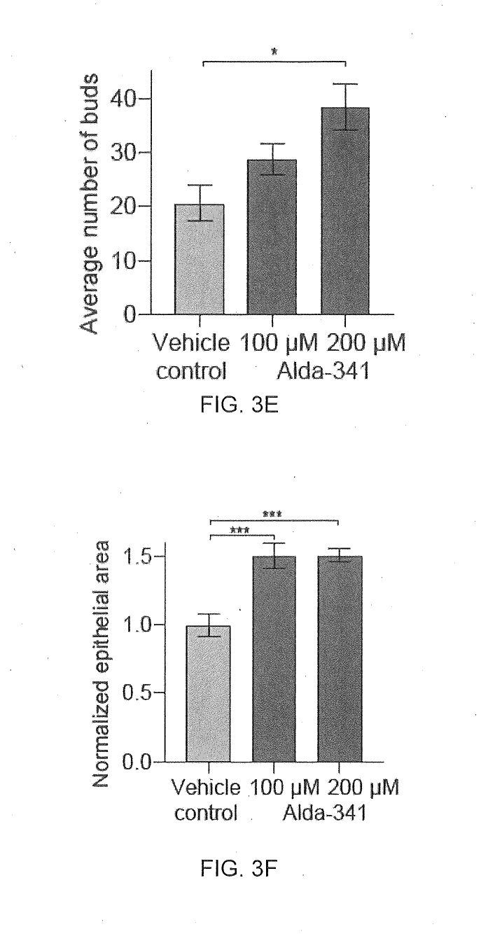

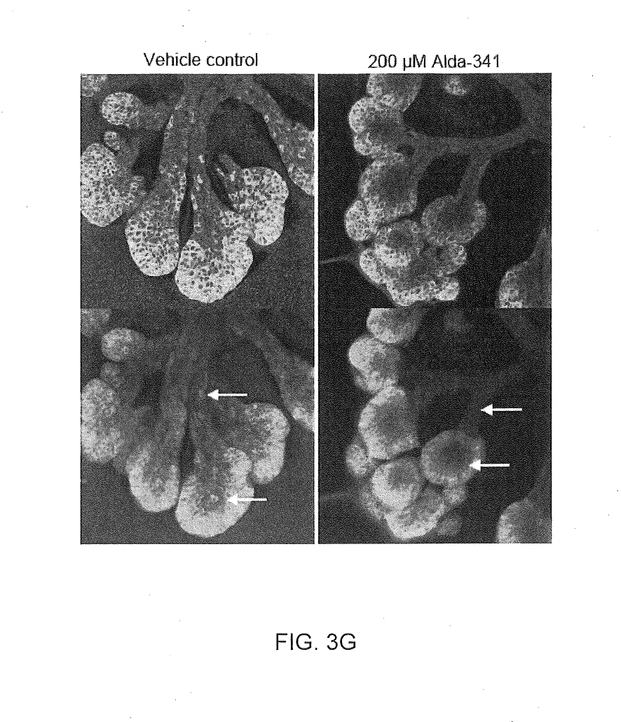

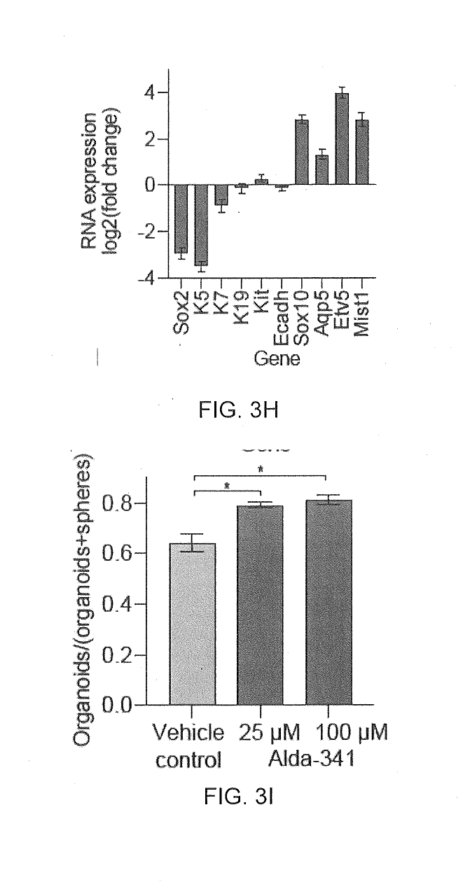

To determine whether activation of ALDH3A1 with Alda-341 increases normal SSPC function, mouse WT EpCAM⁺/CD24⁺-sorted cells were treated with 25 μM Alda-341 over 7 days and about a 15% increase in total salisphere number was observed compared to vehicle control (FIG. 3A).

Human salivary cells dissociated from normal salivary gland tissues that were removed surgically from patients with head and neck cancer treated with 25 μM Alda-341 also formed about 30% more salispheres than untreated cells (FIG. 3, panel B). To determine whether increased sphere formation with Alda-341 may be due in part to decreased apoptosis, 100 μM Alda-341-treated and vehicle control-treated murine WT EpCAM⁺/CD24⁺-sorted cells were treated with Annexin V and propidium iodide and the cells were analyzed with FACS (FIG. 3C). Alda-341-treated cells demonstrated about 60% fewer early and late apoptotic cells compared to vehicle control. These data suggest that Alda-341 can enhance the self-renewal capacity of both mouse and human SSPC likely by reducing apoptosis in this important cell population. To determine whether Alda-341 can also enhance SSPC expansion, E13.5 embryonic SMG epithelia were cultured for 24 h and observed a dose-dependent increase in the number of end buds and epithelial area (FIG. 3D- FIG. 3F) Immunofluorescence staining with c-Kit, a distal progenitor expansion marker, identified c-Kit⁺ cells in both the branches and end buds of the WT epithelia. In contrast, c-Kit⁺ cells in the 200 μM Alda-341-treated epithelia were not found in the branches but rather were limited to the end buds only and appeared structurally organized around the outer rim of the buds (FIG. 3G). These data suggest that these pre-acinar cells were further along in morphological development following Alda-341 treatment. Increased expression of acinar progenitor marker, Sox10, further supports the function of Alda-341 in progenitor cell expansion, and increased transcript levels of acinar maturation markers, Aqp5 and Mist1, is consistent with acinar-specific differentiation (FIG. 3H). To determine whether Alda-341 also enhances differentiation of adult SSPC, the effect of Alda-341 treatment on adult murine organoid formation from salispheres of C57BL/6 mice that were seeded on a surface of collagen and matrigel were measured. Salispheres treated with 25 μM Alda-341 developed 25% more organoids than vehicle-treated control (FIG. 3I).

FIG. 3A shows mouse submandibular gland (SMG) EpCAM+/CD24+ cells sorted by flow cytometry, passaged and grown into spheres for 7 d with 100 μM or 200 μM Alda-341 or vehicle control. Sphere number per well quantified by Image J (NIH). 6 replicates per group.

FIG. 3B shows human salivary cells dissociated from surgically removed human salivary glands, passaged and grown into spheres for 7 d in the presence of vehicle control or 100 μM Alda-341. Sphere number per well quantified by Image J (NIH). 4 replicates per group.

FIG. 3C shows mouse SMG EpCAM+/CD24+ cells sorted by flow cytometry, passaged and treated with 100 μM or vehicle control for 24 h, stained with Annexin V and propidium iodide, and analyzed by flow cytometry for percentage of early and late apoptotic cells. FIG. 3, panel C (right) shows Annexin V⁺PI⁻ are interpreted as early apoptotic cells and appear in the upper left quadrant. Annexin V⁺PI⁺ are interpreted as late apoptotic cells and appear in the upper right quadrant. 5-6 replicates per group.

FIG. 3D shows representative images of E13.5 SMG epithelia from CD-1 mouse embryos were treated with vehicle control, 100 μM, and 200 μM Alda-341 (left to right), cultured for 24 h in the presence of 400 ng/FGF-10 and 0.2 μg/mL HS, and imaged at 10× magnification with brightfield. N=6-7 epithelia per group.

FIG. 3E shows bud number was counted for each epithelia and averaged per group.

FIG. 3F shows epithelial area was quantified using Image J (NIH) and normalized to vehicle control.

FIG. 3G shows representative images for vehicle control (left) and 200 μM Alda-341 (right) after 24 h in culture and immunostained with c-KIT, ECAD, and DAPI, and imaged with a confocal microscope. 1 μM confocal section of all three markers (top) and averaged confocal sections of c-KIT only (bottom).

FIG. 3H shows reverse transcription quantitative PCR of RNA extracted from 4 epithelia per group. RNA expression of epithelia treated with 200 μM Alda-341 represented as a log2 fold change over RNA expression of epithelia treated with vehicle control. Samples were tested in triplicate.

FIG. 3I shows organoids were grown from mouse adult SMG spheres and treated with 25 μM, 100 μM Alda-341 or vehicle control for 7 d. Organoids, defined as having greater than or equal to 5 buds, were blindly counted and quantified as a ratio of organoid number to total number of organoids and undifferentiated spheres. 4 replicates per group. All error bars represent SEM. *=p<0.05; **=p<0.01; ***=p<0.001 (Student's t-test).

### Example 4

### Alda-341 Reduces Aldehydic Load After Radiation in Isolated Embryonic Salivary Glands and Mitigates Radiation-Induced Hyposalivation In Vivo

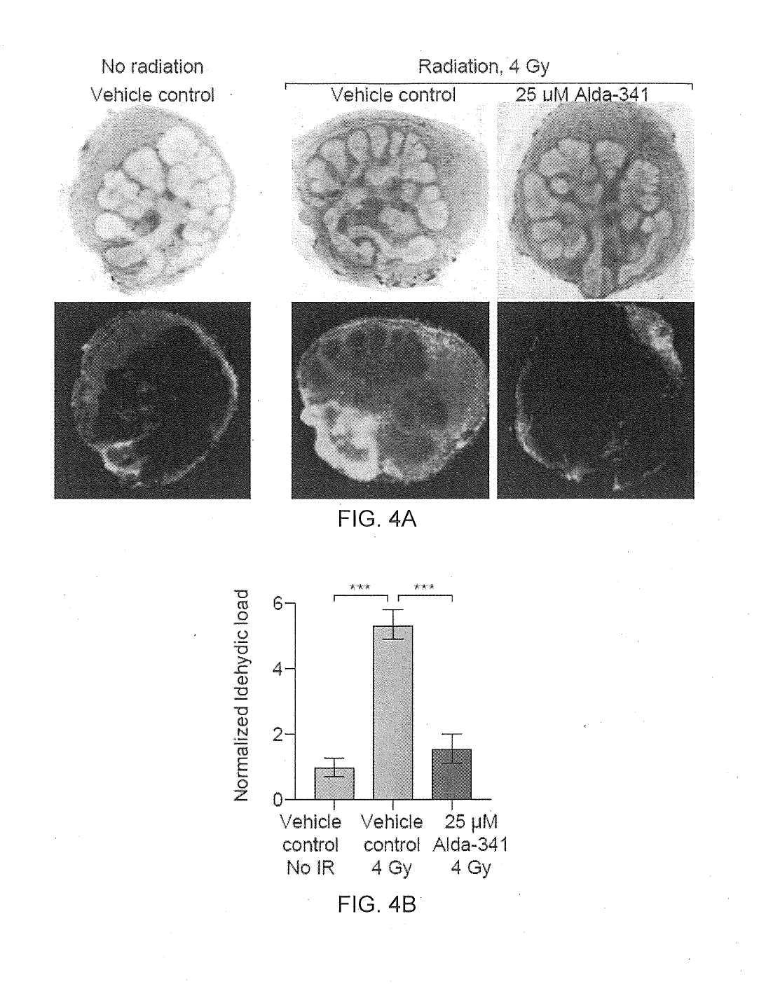

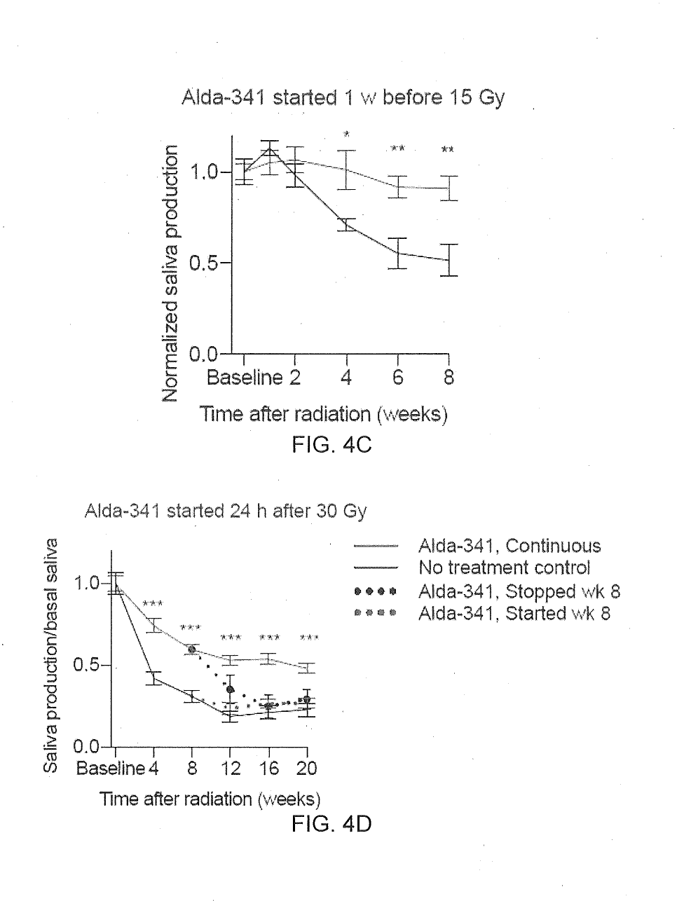

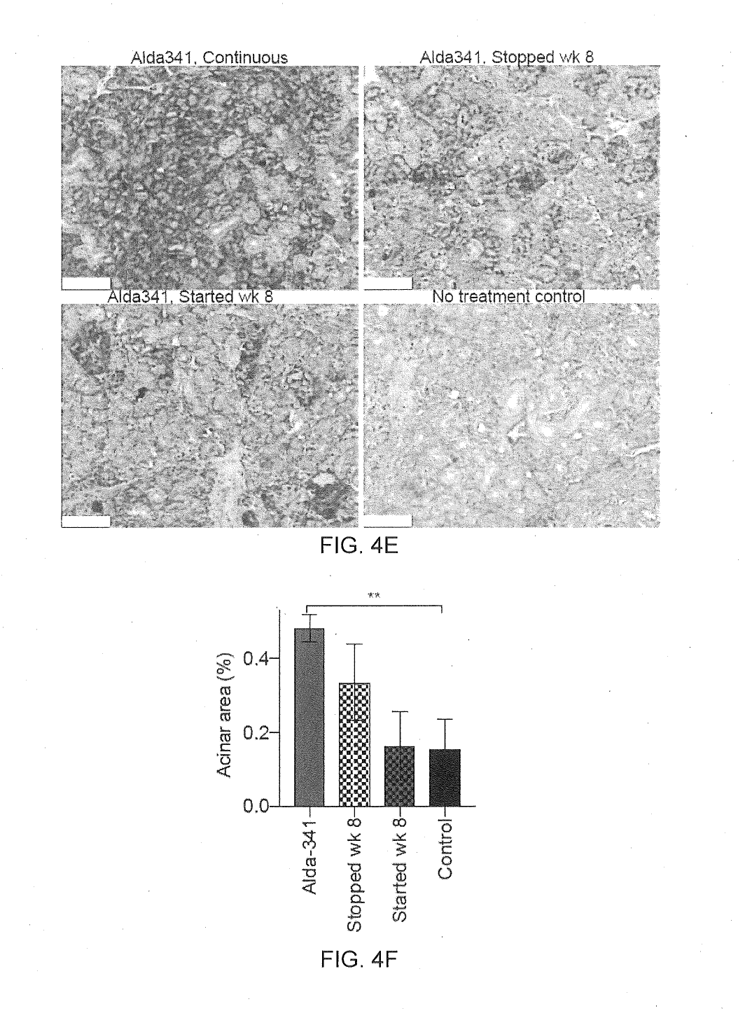

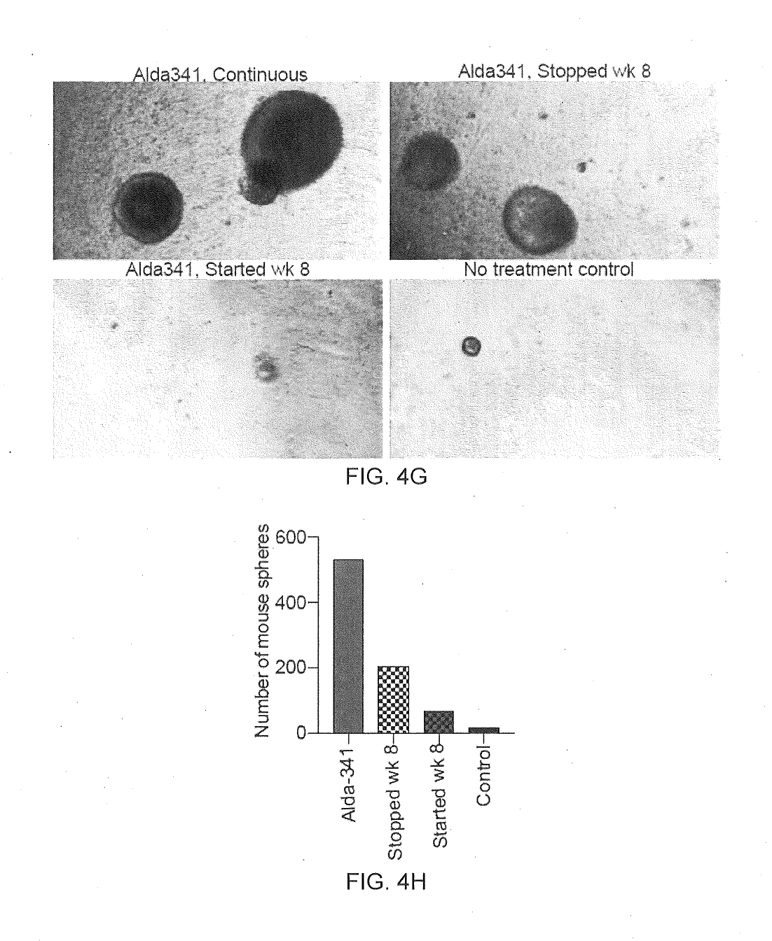

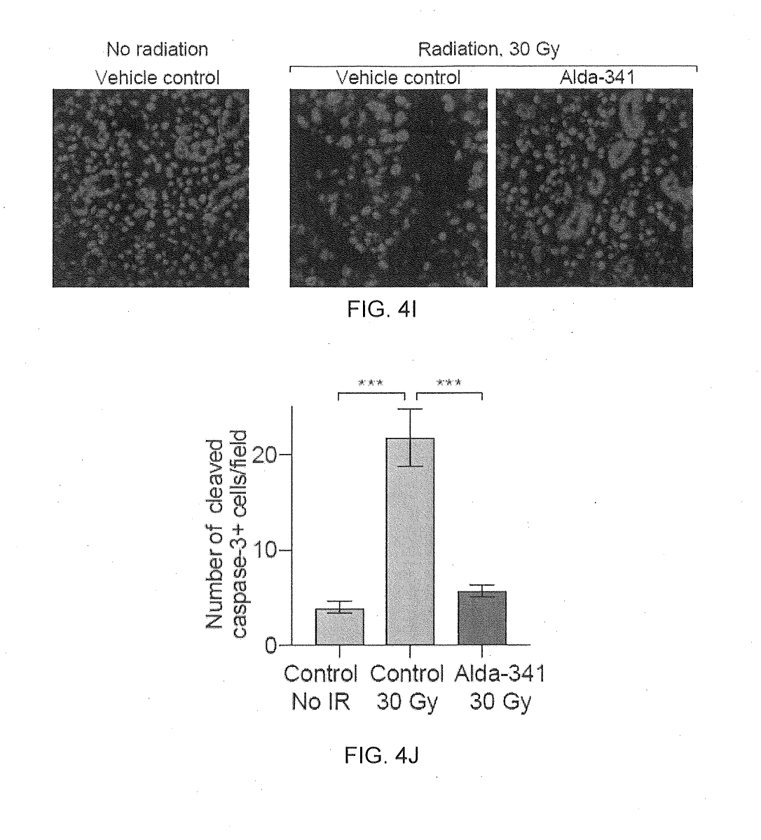

To determine whether Alda-341 is sufficient to reduce radiation injury, intact SMG from CD-1 E13.5 embryos were cultured with 25 μM Alda-341 or with vehicle control. After 24 h, the glands were irradiated with 4 Gy and incubated SMG with DarkZone dye to measure intracellular aldehydes. After 3 h, the aldehydic load in the irradiated vehicle control-treated SMG was more than 4-fold greater than the non-irradiated vehicle control-treated SMG. 25 μM Alda-341-treatment reduced the aldehydic load to almost non-irradiated levels (FIG. 4A-FIG. 4B).

Using a mouse model of radiation-induced hyposalivation, it was next determined whether Alda-341 could reduce injury to the salivary glands after radiation and preserve salivary gland structure and function. Baseline saliva was collected from 8-10 week-old female C57/BL6 mice and began treatment with 10% Alda-341 mixed in mouse chow daily for 1 w prior to radiation and continuously after radiation. Alda-341 levels distributed to mouse SMG measured ˜7000 ng/g by gas chromatography mass spectrometry (GCMS). In one experiment, mice were irradiated with a single dose of 15 Gy and in a second experiment with 6 Gy/d for 5 d for a total of 30 Gy. In both experiments, mice receiving Alda-341 retained near baseline saliva production levels after radiation in contrast to the 50% reduction in mice receiving no treatment (FIG. 4C). Eight weeks after 30 Gy radiation, the Alda-341-treated SMG maintained 90% preservation of acinar area compared with less than 30% for the irradiated control-treated group. To determine whether prophylactic treatment is necessary and to identify the optimal treatment duration, the same experimental design was repeated but Alda-341 treatment started 24 h after the final dose of radiation and continued daily thereafter. Starting 8 weeks after radiation, the control and treatment groups were further segmented into four groups. Half of the control group began daily Alda-341 treatment and half of the treatment group stopped Alda-341. The group that received continuous Alda-341 starting 24 h after the final dose of radiation sustained significantly higher saliva levels over 20 weeks as compared with the other three groups. The group that stopped Alda-341 treatment 8 weeks after radiation experienced a rapid decline in saliva production, and the group that began Alda-341 treatment 8 weeks after radiation did not show significant improvement in saliva production (FIG. 4D). Consistent with these data, Periodic acid Schiff (PAS) staining after 20 weeks, which stains acinar cells positive, showed ˜70% more acinar cell preservation in the group receiving continuous Alda-341 compared with the group that never received Alda-341 (FIG. 4E-FIG. 4F). Likewise, dissociated SMG from mice treated with continuous Alda-341 demonstrated nearly 30-fold improvement in sphere-forming ability compared with mice receiving no treatment, suggesting greater SSPC survival after radiation with continuous Alda-341 treatment (FIG. 4G-FIG. 4H).

On day 5 of the 30 Gy radiation dose, SMG were removed, fixed, and stained for cleaved caspase 3 to determine the extent of apoptosis (FIG. 4E). Irradiated SMG demonstrated a greater than 5-fold increase in cleaved caspase 3 staining as compared with non-irradiated SMG, indicating that apoptosis was already occurring during the course of fractionated radiation. Treatment with Alda-341 before radiation reduced the number of apoptotic cells to near non-irradiated levels (FIG. 4F). This is consistent with RNA-sequencing of EpCAM+ cells isolated from SMG 2 weeks after 30 Gy, which demonstrated an increase in apoptotic-related gene expression after radiation and a decrease in apoptotic-related gene expression after radiation with the treatment of Alda-341. RNA-sequencing also demonstrated a correlation between Alda-341 treatment and increased glutathione metabolism as well as decreased immune response.

A complete blood assessment and necropsy of 3 C57BL/6 female mice irradiated with 30 Gy and treated for 8 weeks with Alda-341 and 3 irradiated control mice showed normal complete blood count and blood chemistry panel and no organ toxicity. In a separate xenograft study of 6-week old SCID mice implanted with SAS (human papillomavirus [HPV]-negative) and SSC90 (HPV-positive) head and neck squamous cell carcinoma cell lines, Alda-341 treatment did not increase tumor growth or protect tumors from radiation. Together, these data suggest that Alda-341 is safe for this indication.

### Example 5

### Alda-341 Distributed Into Human Salivary Glands

A Phase 0 study was conducted at Stanford Hospital and Clinics (Stanford, Calif.), to determine whether Alda-341 is distributed into the salivary gland and saliva. Patients who were scheduled to undergo surgical removal of a salivary gland tumor (either benign or malignant) were given 2 g daily (1 g twice a day) of oral Alda-341 daily for 2 weeks immediately before their scheduled surgery. Saliva and plasma samples were collected at baseline, and at the time of surgery, and normal salivary gland tissue was collected at the time of surgery. Alda-341 levels were measured in plasma, saliva, and salivary gland tissue by GCMS.

Phase 0 study in head and neck cancer patients: This study was approved by Stanford University's Institutional Review Board. Patients diagnosed with salivary gland tumors and scheduled to undergo salivary gland surgery were recruited for this 2-week, open-label, oral disposition study of Alda-341. Patients were required to be between 18 to 85 years of age, have elected to undergo surgery for recent diagnosis of parotid or submandibular gland tumor, and qualified for anesthesia. Written informed consent was obtained from all patients before any study procedure was conducted. Patients were excluded from study participation if nursing or pregnant or diagnosed with kidney disease, end stage liver disease, metastatic cancer, or any unstable medical condition. Four subjects began Alda-341 treatment at 2 g/day (1 g twice per d) for 14 d prior to surgery. Blood and saliva samples were collected at baseline and on the day of surgery. Normal and tumor salivary gland tissue were collected during surgery. Drug levels in tissue, plasma, and saliva were measured by the GCMS method described herein.

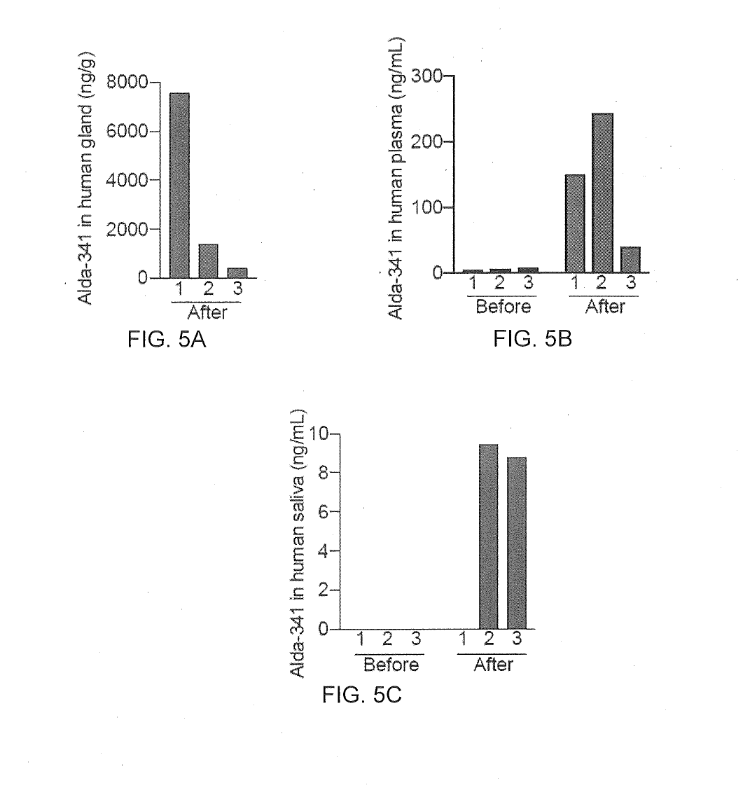

Data collected from the first 3 patients showed that Alda-341 concentrates at higher levels in salivary gland tissues than saliva and blood, possibly because of the compound's hydrophobic properties. Salivary gland drug levels measured on average 3156.7 ng/g (FIG. 5A), which is similar in order of magnitude to drug levels measured in murine salivary glands after 2 weeks of 10% Alda-341 delivered in mouse chow. Average plasma drug levels measured 6.0 ng/mL before and 144.3 ng/mL after treatment (FIG. 5B). Average saliva drug levels measured 0 ng/mL before treatment and 6.1 ng/mL after treatment (FIG. 5C).

FIG. 5A shows Alda-341 levels in human salivary gland tissue measured with GCMS after 2 weeks of 2 g/d Alda-341 oral treatment. 3 samples from different parts of the gland tissue per patient were analyzed.

FIG. 5B shows Alda-341 levels in human plasma measured with GCMS at baseline and after 2 weeks of 2 g/d Alda-341 oral treatment. One sample per time point was analyzed.

FIG. 5C shows Alda-341 levels in human saliva measured with GCMS at baseline and after 2 weeks of 2 g/d Alda-341 oral treatment. Two samples per time point were analyzed and represented as an average.

### Materials and Methods

Drugs: A library of traditional Chinese medicinal plants were donated by Sun Ten Pharmaceuticals Co. in Taiwan. Alda-341 (D-limonene) and all other screened compounds, PEG-400, and pilocarpine were purchased from Sigma-Aldrich. Isoflurane (VetOne), ketamine (VEDCO) and xylazine (AnaSEd) were acquired through Stanford University's Veterinary Service Center.

Animals: C57BL/6 wild-type mice were purchased from Jackson Labs and C57BL/6 Aldh3a1 were obtained from the laboratory of Vasilis Vasiliou at Yale School of Public Health, New Haven, Conn. (33). Timed pregnant CD-1/ICR mice were purchased from Envigo (Cat #044). The Administrative Panel on Laboratory Animal Care at Stanford University, Stanford, Calif. and the Institutional Animal Care and Use Committee at University of California, San Francisco, Calif. approved all animal protocols. Mice were kept at 20-26° C., 30-70% humidity, in a 12 hour light/dark cycle. All animal experimentation was conducted in adherence to the NIH Guide for the Care of and Use of Laboratory Animals.

Saliva collection: Female C57/BL-6 mice, 8-10 weeks old, were ordered from Jackson Labs. Mice were treated with 10% Alda-341 mixed in chow or no treatment. 15 Gy single dose or 30 Gy fractionated over 5 d (6 Gy/d) were delivered to the SMG with the rest of the body lead shielded. Stimulated saliva was measured as described in, Lombaert, I. M., Brunsting, J. F., Wierenga, P. K., Faber, H., Stokman, M. A., Kok, T., Visser, W. H., Kampinga, H. H., de Haan, G., and Coppes, R. P. (2008) Rescue of salivary gland function after stem cell transplantation in irradiated glands. PloS one 3, e2063. Mice were anesthetized with a ketamine (80 mg/kg) and xylazine (16 mg/kg) mixture delivered by intraperitoneal injection and subcutaneously injected with 2 mg/kg pilocarpine. Saliva was collected for 15 minutes. Saliva volume was calculated by assuming that 1 and was normalized to the mouse body weight by dividing the total collected saliva volume by the mass of the mouse (kg).

Salivary gland tissue dissociation: Mouse and human salivary gland cells were isolated as previously described in, Szlavik, V., Szabo, B., Vicsek, T., Barabas, J., Bogdan, S., Gresz, V., Varga, G., O'Connell, B., and Vag, J. (2008) Differentiation of primary human submandibular gland cells cultured on basement membrane extract. Tissue engineering. Part A 14, 1915-1926. Mouse glands were homogenized and incubated in DMEM/F12 with collagenase (0.025%) and hyaluronidase (0.04%) (Stem Cell Technologies, 07912), 6.25 mM CaCl, and antifungal (Omega scientific; 1:500) for 1 hour and in dispase (BD Biosciences) for 1 hour on a shaker at 37° C. Tissue was filtered through 100 μM cell strainer and centrifuged at 1200 rpm for 6 minutes. Red blood cells were lysed with ACK Lysing Buffer (Lonza, 10-548E) for 2 minutes, inactivated with 10% FBS DMEM, filtered through 100 μM cell strainer, and centrifuged at 1200 rpm for 6 minutes. Cells were then trypsinized with 0.25% trypsin for 1 minute, inactivated with 10% FBS DMEM, filtered through 40 μM cell strainer, and centrifuged at 1200 rpm for 6 minutes.

Annexin V assay: EpCAM+CD24+ sorted salivary sphere cells were grown in matrigel as described above and treated with vehicle control (PEG-400) or 100 μM Alda-341 for 48 hours. Cells were dissociated from matrigel, stained with Biolegend's FITC Annexin V Apoptosis Detection Kit with Propidium Iodide (Life Technologies), and analyzed by flow cytometry. Annexin V⁺PI⁻ cells were analyzed as early apoptotic cells and Annexin V⁺PI⁺ cells were analyzed as late apoptotic cells. Five to six replicates per group were assayed.

Immunohistochemistry: Embryonic rudiment immunofluorescence analysis has been previously described in, Knox, S. M., Lombaert, I. M., Reed, X., Vitale-Cross, L., Gutkind, J. S., and Hoffman, M. P. (2010) Parasympathetic innervation maintains epithelial progenitor cells during salivary organogenesis. Science (New York, N.Y.) 329, 1645-1647. In brief, tissue was fixed with 4% PFA for 20-30 min followed by permeabilizing with 0.1-0.3% Triton-X. Tissue was blocked overnight at 4° C. with 10% Donkey Serum (Jackson Laboratories), 1% BSA (Sigma Aldrich), and MOM IgG-blocking reagent (Vector Laboratories) in 0.01% PBS-Tween-20. SGs were incubated with primary antibodies overnight at 4° C.: rabbit anti-cKIT (1:200, Santa Cruz Biotech, M14), and rat anti-E-cadherin (1:300, Life Technologies, 13-1900). Antibodies were detected using Cy2-, Cy3- or Cy5-conjugated secondary Fab fragment antibodies (Jackson Laboratories) and nuclei stained using Hoescht 33342 (1:1000, Sigma Aldrich). Fluorescence was analyzed using a Leica Sp5 confocal microscope and NIH ImageJ software.

qPCR: RNA was isolated from whole tissue using RNAqueous Micro Kit (Ambion). Total RNA samples were DNase-treated (Ambion), prior to cDNA synthesis using SuperScript reagents (Invitrogen). SYBRgreen qPCR was performed using 1 ng of cDNA and primers designed using Primer3 and Beacon Designer software or found using PrimerBank (http://pga.mgh.harvard.edu/primerbank/). Melt-curves and primer efficiency were determined as previously described (Hoffman, M. P., Kidder, B. L., Steinberg, Z. L., Lakhani, S., Ho, S., Kleinman, H K., and Larsen, M. (2002) Gene expression profiles of mouse submandibular gland development: FGFR1 regulates branching morphogenesis in vitro through BMP- and FGF-dependent mechanisms. Development (Cambridge, England) 129, 5767-5778). Gene expression was normalized to the housekeeping gene S29 (Rps29).

Organoid formation assay: Spheres were dissociated in 1 mg/mL dispase (BD Biosciences) for 20 minutes and resuspended in 80% collagen, 10% 10× F12, and 10% Solution C (2.2 g NaHCO₃ in 100 mL of 0.05 N NaOH and 200 mM HEPES) and combined with 60% matrigel. 200 spheres per well were plated in a Millicell cell culture insert (EMD Millipore) with sphere media. Organoids defined as having greater than or equal to 5 buds formed after 2-3 d in culture and were grown for 7 d before imaging with a BZ-X710 Keyence microscope using the z-stack function. Images were stitched together into a single image and the number of differentiated organoids and undifferentiated spheres were blindly counted and quantified as a ratio of organoid number to total number of organoids and undifferentiated spheres. Experiments were repeated at least three independent times.

Gas chromatography mass spectrometry: Human salivary gland samples were prepared using 25% tissue homogenate in phosphate buffer, spiked with 10,000 ng/mL perillyl aldehyde (in acetonitrile) as an internal standard, and extracted with heptane. An aliquot of the heptane layer was used for analysis. Alda-341 concentrations were measured using an Agilent 7890/5975C GC/MSD equipped with an Agilent 7693 autosampler. Samples volumes of 1 μL splitless were injected with an inlet temperature of 220° C. The flow rate was constant at 1 mL/min. An Agilent DB-5MS UI column was used (30 m length, 0.250 mm inner diameter, 0.25 micron film). The temperature was held at 60° C. for 1 min, increased to 150° C. at a rate of 30° C./min, increased to 320° C. at 60° C./min and held for 3.167 min for a total run time of 10 min. Calibration curves were linear from 4 to 8000 ng/mL.

Periodic acid schiff staining and acinar quantification: SMG were removed from mice, fixed in 10% formalin for 24 h, and paraffin-embedded. 10 images per group were taken at random at 30× magnification using a Leica DM6000 B microscope. Acinar regions were quantified using RT_Image software (Graves, E. E., Quon, A., and Loo, B. W., Jr. (2007) RT_Image: an open-source tool for investigating PET in radiation oncology. Technology in cancer research & treatment 6, 111-121). Images were separated into red, green, and blue color channels, blurred using a 20 pixel box filter, and segmented using an intensity contour of 110 with a minimum area of 100 um². Acinar area was calculated as a percentage of acinar area relative to the area of the microscopy field.

Cleaved caspase 3 staining: SMG were removed from mice, fixed in 10% formalin for 24 h, and paraffin-embedded. Sections were stained with Caspase 3 rabbit antibody (Cell Signaling, 9664), 1:200 dilution, and DAPI. Three random images using red and blue filters were taken at 100× magnification with a Leica DM6000 B microscope from each gland (6-8 glands/group) and quantified by counting cleaved caspase-3 positive (red) cells per field.

RNA-sequencing: Samples were extracted using Qiagen miRNeasy Kit (217084). Extracted samples were assessed for quality using Agilent Pico-RNA bio-analyzer chip (5067-1513). The Smarter Ultra Low Input RNA kit (Clontech, 634848) was used to generate cDNA from total RNA. Amplified cDNA was purified using SPRI Ampure Beads from Beckman Coulter, and quality and quantity were measured using a High Sensitivity DNA chip on an Agilent 2100 Bioanalyzer. cDNA was sheared to an average length of 300 BP using a Covaris S2, and libraries were generated following the Clontech Low Input Library Prep kit (634947). Indexed libraries were pooled and quantitated for sequencing. Sequencing data was generated on an Illumina HiSeq 4000. The processed RNAseq reads were imported into BRB-ArrayTools, an integrated package for the visualization and statistical analysis of gene expression data developed by Dr. Richard Simon and BRB-ArrayTools Development Team. The imported expression values were log2-transformed and subjected to quantile normalization by Robust Multi-chip Average (RMA). Genes that show differential expression between those untreated and irradiated (univariate p-value <0.01) were selected. To identify functional networks and pathways enriched in these significant genes, these genes were analyzed using MetaCore™. For heatmap generation the Clustering function of ArrayTools was used, with experimental samples in fixed grouping and order, and genes ordered by hierarchical clustering. Color scales of the individual heatmaps are shown on the side. From these heatmaps, genes that share similar differential expression patterns were identified.

Pathology: A complete necropsy was performed by a certified veterinarian. Tissues from brain, heart, kidney, liver, esophagus, thymus, trachea, thyroid, lung, spleen, pancreas, trachea, stomach, pancreas, intestines, bladder, uterus and ovary were fixed on slides and scored for inflammation, necrosis, and apoptosis from a scale of 0 to 5. A complete blood count and blood chemistry panel was assessed by Stanford's Veterinary Services Center.

Xenograft study: Six-week old SCID mice were purchased from the Jackson laboratory and five mice per group were used. SCC90 and SAS cells (2×10⁶ cells/injection) were implanted into both flanks of each mouse. Alda-341 treated one week ahead of irradiation and continued during and post radiation. Total 30 Gy over 5 fractions in a week was delivered to the tumor with the rest of the body shielded. Tumor size was measured every 1-2 days. Tumor volume was calculated by the formula (π×length×width×height)/6.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

Notwithstanding the appended claims, the disclosure set forth herein is also described by the following clauses.

Clause 1. A method of increasing proliferation of adult salivary stem cells, the method comprising contacting a starting population of adult salivary stem cells in vitro, in vivo, or ex vivo with a therapeutically effective amount of at least one isolated monoterpene; and subjecting the adult salivary stem cells to radiation treatment before or after said contacting, wherein after the contacting and the radiation treatment the number of adult salivary stem cells increases by 25% or more.

Clause 2. The method of clause 1, wherein said contacting is in vivo, and wherein said contacting comprises administering an effective amount of said at least one isolated monoterpene to an individual in need thereof.

Clause 3. The method of clause 2, wherein said individual has a head and neck cancer and has undergone or is about to undergo radiation treatment for the head and neck cancer.

Clause 4. The method of clause 1, wherein said contacting is ex vivo, and wherein said contacting generates an expanded population of adult salivary stem cells.

Clause 5. The method of clause 4, further comprising introducing the expanded population of adult salivary stem cells into a recipient individual, wherein the recipient individual has a head and neck cancer, and wherein said introducing occurs after the recipient individual has undergone radiation treatment for the cancer.

Clause 6. The method of clause 5, wherein the expanded adult salivary stem cells are expanded using adult salivary stem cells obtained from the recipient individual before the individual undergoes radiation treatment.

Clause 7. The method of clause 5, wherein the expanded population of adult salivary stem cells is expanded from adult salivary stem cells obtained from an individual other than the recipient individual.

Clause 8. The method of any one of clauses 5 to 7, wherein the recipient individual is a human.

Clause 9. The method of any one of clauses 1 to 8, wherein the isolated monoterpene is an ALDH3 agonist.

Clause 10. The method of any one of clauses 1 to 9, wherein the isolated monoterpene is selected from the group consisting of, R)-(+)-limonene, (S)-(−)-limonene, alpha-pinene, beta-pinene, alpha-terpinene, gamma-terpinene, terpineol, alpha-phellandrene, beta phellandrene, (S)-(−)-perillyl alcohol and (R)-(+)-perillyl alcohol, or a pharmaceutically acceptable salt thereof.

Clause 11. The method of clause 10, wherein the isolated monoterpene is (R)-(+)-limonene or a pharmaceutically acceptable salt thereof.

Clause 12. The method of clause 1, further comprising contacting said adult salivary stem cells in vitro with an antibody that specifically recognizes a marker associated with adult salivary stem cells, wherein contacting said adult salivary stem cells with the antibody is performed prior to contacting said adult salivary stem cells with said isolated monoterpene.

Clause 13. The method of clause 12, wherein the marker is selected from CD34, CD90, c-Kit, CD44, Nestin, and combinations thereof.

Clause 14. A method of treating an individual having a head and neck cancer, the method comprising:
a) subjecting the individual to radiation therapy for the head and neck cancer; and
b) administering to the individual an isolated monoterpene in an amount effective to increase the number of adult salivary stem cells in the individual, wherein said administering increases the number of functioning saliva-producing cells in the individual.

Clause 15. The method of clause 14, further comprising administering to the individual an effective amount of an isolated monoterpene before subjecting the individual to radiation therapy for the head and neck cancer.

Clause 16. The method of clause 14 or 15, wherein the administration of an effective amount of said at least one isolated monoterpene is continuous.

Clause 17. The method of clause 16, wherein at least one monoterpene is administered continuously 1 week or more before radiation treatment for the head and neck cancer and 8 weeks or more after radiation treatment for the head and neck cancer.

Clause 18. A method of protecting adult salivary stem cells, the method comprising contacting a starting population of adult salivary stem cells in vitro, in vivo, or ex vivo with a therapeutically effective amount of at least one isolated monoterpene; and subjecting the adult salivary stem cells to radiation treatment before or after said contacting, wherein after the contacting and the radiation treatment the adult salivary stem cells are protected from radiation injury relative to the starting population of adult salivary stem cells.

Clause 19. The method of clause 18, wherein said contacting is in vivo, and wherein said contacting comprises administering an effective amount of said at least one isolated monoterpene to an individual in need thereof.

Clause 20. The method of clause 19, wherein said individual has a head and neck cancer and has undergone or is about to undergo radiation treatment for the head and neck cancer.

Clause 21. The method of clause 18, wherein said contacting is ex vivo, and wherein said contacting generates a protected population of adult salivary stem cells.

Clause 22. The method of clause 21, further comprising introducing the protected population of adult salivary stem cells into a recipient individual, wherein the recipient individual has a head and neck cancer, and wherein said introducing occurs after the recipient individual has undergone radiation treatment for the cancer.

Clause 23. The method of clause 22, wherein the protected adult salivary stem cells are generated using adult salivary stem cells obtained from the recipient individual before the individual undergoes radiation treatment.

Clause 24. The method of clause 22, wherein the protected population of adult salivary stem cells is generated from adult salivary stem cells obtained from an individual other than the recipient individual.

Clause 25. The method of any one of clauses 22 to 24, wherein the recipient individual is a human.

Clause 26. The method of any one of clauses 18 to 25, wherein the isolated monoterpene is an ALDH3 agonist.

Clause 27. The method of any one of clauses 18 to 26, wherein the isolated monoterpene is selected from the group consisting of, R)-(+)-limonene, (S)-(−)-limonene, alpha-pinene, beta-pinene, alpha-terpinene, gamma-terpinene, terpineol, alpha-phellandrene, beta phellandrene, (S)-(−)-perillyl alcohol and (R)-(+)-perillyl alcohol, or a pharmaceutically acceptable salt thereof.

Clause 28. The method of clause 27, wherein the isolated monoterpene is (R)-(+)-limonene or a pharmaceutically acceptable salt thereof.

Clause 29. The method of clause 18, further comprising contacting said adult salivary stem cells in vitro with an antibody that specifically recognizes a marker associated with adult salivary stem cells, wherein contacting said adult salivary stem cells with the antibody is performed prior to contacting said adult salivary stem cells with said isolated monoterpene.

Clause 30. The method of clause 29, wherein the marker is selected from CD34, CD90, c-Kit, CD44, Nestin, and combinations thereof.

Clause 31. A method of treating an individual having a head and neck cancer, the method comprising:
a) subjecting the individual to radiation therapy for the head and neck cancer; and
b) administering to the individual an isolated monoterpene in an amount effective to protect the adult salivary stem cells in the individual, wherein said administering protects functioning saliva-producing cells in the individual.

Clause 32. The method of clause 31, further comprising administering to the individual an effective amount of an isolated monoterpene before subjecting the individual to radiation therapy for the head and neck cancer.

Clause 33. The method of clause 31 or 32, wherein the administration of an effective amount of said at least one isolated monoterpene is continuous.

Clause 34. The method of clause 33, wherein at least one monoterpene is administered continuously 1 week or more before radiation treatment for the head and neck cancer and 8 weeks or more after radiation treatment for the head and neck cancer.

Clause 35. A method improving salivary gland function, the method comprising contacting a starting population of adult salivary stem cells in vitro, in vivo, or ex vivo with a therapeutically effective amount of at least one isolated monoterpene; and subjecting the adult salivary stem cells to radiation treatment before or after said contacting, wherein after the contacting and the radiation treatment the adult salivary stem cells have improved salivary gland function relative to the starting population of adult salivary stem cells.

Clause 36. The method of clause 35, wherein said contacting is in vivo, and wherein said contacting comprises administering an effective amount of said at least one isolated monoterpene to an individual in need thereof.

Clause 37. The method of clause 36, wherein said individual has a head and neck cancer and has undergone or is about to undergo radiation treatment for the head and neck cancer.

Clause 38. The method of clause 35, wherein said contacting is ex vivo, and wherein said contacting generates a population of adult salivary stem cells having improved salivary gland function.

Clause 39. The method of clause 38, further comprising introducing the population of adult salivary stem cells having improved salivary gland function into a recipient individual, wherein the recipient individual has a head and neck cancer, and wherein said introducing occurs after the recipient individual has undergone radiation treatment for the cancer.

Clause 40. The method of clause 39, wherein the adult salivary stem cells having improved salivary gland function are generated using adult salivary stem cells obtained from the recipient individual before the individual undergoes radiation treatment.

Clause 41. The method of clause 39, wherein the population of adult salivary stem cells having improved salivary gland function is generated from adult salivary stem cells obtained from an individual other than the recipient individual.

Clause 42. The method of any one of clauses 39 to 41, wherein the recipient individual is a human.

Clause 43. The method of any one of clauses 35 to 42, wherein the isolated monoterpene is an ALDH3 agonist.

Clause 44. The method of any one of clauses 35 to 43, wherein the isolated monoterpene is selected from the group consisting of, R)-(+)-limonene, (S)-(−)-limonene, alpha-pinene, beta-pinene, alpha-terpinene, gamma-terpinene, terpineol, alpha-phellandrene, beta phellandrene, (S)-(−)-perillyl alcohol and (R)-(+)-perillyl alcohol, or a pharmaceutically acceptable salt thereof.

Clause 45. The method of clause 44, wherein the isolated monoterpene is (R)-(+)-limonene or a pharmaceutically acceptable salt thereof.

Clause 46. The method of clause 35, further comprising contacting said adult salivary stem cells in vitro with an antibody that specifically recognizes a marker associated with adult salivary stem cells, wherein contacting said adult salivary stem cells with the antibody is performed prior to contacting said adult salivary stem cells with said isolated monoterpene.

Clause 47. The method of clause 46, wherein the marker is selected from CD34, CD90, c-Kit, CD44, Nestin, and combinations thereof.

Clause 48. A method of treating an individual having a head and neck cancer, the method comprising:
a) subjecting the individual to radiation therapy for the head and neck cancer; and
b) administering to the individual an isolated monoterpene in an amount effective to improve the salivary gland function of the adult salivary stem cells in the individual, wherein said administering improves functioning saliva-producing cells in the individual.

Clause 49. The method of clause 48, further comprising administering to the individual an effective amount of an isolated monoterpene before subjecting the individual to radiation therapy for the head and neck cancer.

Clause 50. The method of clause 48 or 49, wherein the administration of an effective amount of said at least one isolated monoterpene is continuous.

Clause 51. The method of clause 50, wherein at least one monoterpene is administered continuously 1 week or more before radiation treatment for the head and neck cancer and 8 weeks or more after radiation treatment for the head and neck cancer.

Clause 52. A method of treating dry eye, the method comprising administering a therapeutically effective amount of at least one isolated monoterpene to an individual in need thereof.

Clause 53. The method of clause 52, wherein the monoterpene is selected from the group consisting of, (R)-(+)-limonene, (S)-(−)-limonene, alpha-pinene, beta-pinene, alpha-terpinene, gamma-terpinene, terpineol, alpha-phellandrene, beta phellandrene, (S)-(−)-perillyl alcohol and (R)-(+)-perillyl alcohol or a pharmaceutically acceptable salt thereof.

Clause 54. The method of clause 53, wherein the isolated monoterpene is (R)-(+)-limonene or a pharmaceutically acceptable salt thereof.

What is claimed is:

1. A method of treating adult salivary stem cells, the method comprising:
contacting a starting population of adult salivary stem cells in vitro, in vivo, or ex vivo with a therapeutically effective amount of at least one isolated monoterpene; and
subjecting the adult salivary stem cells to radiation treatment before or after said contacting, wherein the contacting results in one or more of increasing proliferation of the adult salivary stem cells, protecting the adult salivary stem cells from the radiation treatment and improving the salivary gland function of the adult salivary stem cells relative to the starting population of adult salivary stem cells.

2. The method of claim 1, wherein after the contacting and the radiation treatment the number of adult salivary stem cells increases by 25% or more.

3. The method of claim 1, wherein said contacting is in vivo, and wherein said contacting comprises administering an effective amount of said at least one isolated monoterpene to an individual in need thereof.

4. The method of claim 3, wherein said individual has a head and neck cancer and has undergone or is about to undergo radiation treatment for the head and neck cancer.

5. The method of claim 1, wherein said contacting is ex vivo, and wherein said contacting generates a treated population of adult salivary stem cells.

6. The method of claim 5, further comprising introducing the treated population of adult salivary stem cells into a recipient individual, wherein the recipient individual has a head and neck cancer, and wherein said introducing occurs after the recipient individual has undergone radiation treatment for the cancer.

7. The method of claim 6, wherein the treated adult salivary stem cells are generated using adult salivary stem cells obtained from the recipient individual before the individual undergoes radiation treatment.

8. The method of claim 6, wherein the treated population of adult salivary stem cells is generated from adult salivary stem cells obtained from an individual other than the recipient individual.

9. The method of claim 6, wherein the recipient individual is a human.

10. The method of claim 1, wherein the isolated monoterpene is an ALDH3 agonist.

11. The method of claim 1, wherein the isolated monoterpene is selected from the group consisting of, R)-(+)-limonene, (S)-(−)-limonene, alpha-pinene, beta-pinene, alpha-terpinene, gamma-terpinene, terpineol, alpha-phellandrene, beta phellandrene, (S)-(−)-perillyl alcohol and (R)-(+)-perillyl alcohol, or a pharmaceutically acceptable salt thereof.

12. The method of claim 11, wherein the isolated monoterpene is (R)-(+)-limonene or a pharmaceutically acceptable salt thereof.

13. The method of claim 1, further comprising contacting said adult salivary stem cells in vitro with an antibody that specifically recognizes a marker associated with adult salivary stem cells, wherein contacting said adult salivary stem cells with the antibody is performed prior to contacting said adult salivary stem cells with said isolated monoterpene.

14. The method of claim 13, wherein the marker is selected from CD34, CD90, c-Kit, CD44, Nestin, and combinations thereof.

15. A method of treating an individual having a head and neck cancer, the method comprising:
a) subjecting the individual to radiation therapy for the head and neck cancer; and
b) administering to the individual an isolated monoterpene in an amount effective to increase the number of adult salivary stem cells in the individual, wherein said administering increases the number of functioning saliva-producing cells in the individual.

16. The method of claim 15, further comprising administering to the individual an effective amount of an isolated monoterpene before subjecting the individual to radiation therapy for the head and neck cancer.

17. The method of claim 15, wherein the administration of an effective amount of said at least one isolated monoterpene is continuous.

18. The method of claim 16, wherein at least one monoterpene is administered continuously 1 week or more before radiation treatment for the head and neck cancer and 8 weeks or more after radiation treatment for the head and neck cancer.

19. A method of treating dry eye, the method comprising administering a therapeutically effective amount of at least one isolated monoterpene to an individual in need thereof.

20. The method of claim 19, wherein the monoterpene is selected from the group consisting of, (R)-(+)-limonene, (S)-(−)-limonene, alpha-pinene, beta-pinene, alpha-terpinene, gamma-terpinene, terpineol, alpha-phellandrene, beta phellandrene, (S)-(−)-perillyl alcohol and (R)-(+)-perillyl alcohol or a pharmaceutically acceptable salt thereof.

21. The method of claim 20, wherein the isolated monoterpene is (R)-(+)-limonene or a pharmaceutically acceptable salt thereof.
